(19) United States
(12) Patent Application Publication
(10) Pub. No.: US 2024/0050547 A1
(43) Pub. Date: Feb. 15, 2024

(54) METHODS OF BLOCKING / NEUTRALIZING ASFV INFECTION THROUGH INTERRUPTION OF CELLULAR AND VIRAL RECEPTOR INTERACTIONS

(71) Applicants: Dalu CHEN, Erie, CO (US); Thomas MALCOLM, Andover, NJ (US)

(72) Inventors: Dalu CHEN, Erie, CO (US); Thomas MALCOLM, Andover, NJ (US)

(21) Appl. No.: 18/072,783

(22) Filed: Dec. 1, 2022

Related U.S. Application Data
(63) Continuation-in-part of application No. 17/535,545, filed on Nov. 24, 2021.
Application No. 17/535,545 is a continuation-in-part of application No. PCT/US20/50939, filed on Sep. 16, 2020.
(60) Provisional application No. 62/900,816, filed on Sep. 16, 2019.

(51) Int. Cl. A61K 39/12; C07K 14/705; A61K 48/00; C07K 16/08

(52) U.S. Cl. CPC A61K 39/12; C07K 14/70503; A61K 48/0066; C07K 16/081; C07K 2317/76; C07K 2317/52; C07K 2317/31; C07K 2317/14; C07K 2319/30

(57) ABSTRACT

A method of preventing and treating viral infections in animals (and preferably ASFV in porcine), by inhibiting viral ligand interactions with critical cellular receptors that are involved either directly (endo/pinocytosis)) or indirectly (infection through RBCs that have been aggregated by viral interactions) with cellular entry in an animal and preventing and treating the viral infection in the animal. A method of treating a viral infection in an individual with a virus that is both lysogenic and lytic. A composition for treating a viral infection in an individual with a virus that is both lysogenic and lytic. A vaccine for preventing viral infection, including whole and/or partial domains of proteins of both a lysogenic and lytic phase of a virus.

### REFERENCE TO AN ELECTRONIC SEQUENCE LISTING

The contents of the electronic sequence listing (0431.54.Sequence listing.092223_ST25.txt; Size: 6,772 bytes; and Date of Creation: Sep. 22, 2023) is herein incorporated by reference in its entirety.

### BACKGROUND OF THE INVENTION

### 1. Technical Field

The present invention relates to methods and/or treatments for preventing viral infections in animals (non-human). More specifically, the present invention relates to methods of treating and preventing viral infections in swine and other animals.

### 2. Background Art

African swine fever virus (ASFV) is a large double stranded DNA virus that primarily infects domestic pigs, wild boars, warthogs, and bush pigs. It also resides in soft ticks, thereby acting as an infectious vector. ASFV primarily infects the monocytes and macrophages, although, at acute infection many other cell types can be infected. ASFV causes high fever, hemorrhagic lesions, cyanosis, anorexia, and fatalities in these animals. There is no vaccine or treatment for this virus, and the only way to currently prevent its spread is culling animals.

U.S. Provisional Patent Application No. 62/871,949 to Applicants discloses a gene drive for eliminating or neutralizing virus carriers such as soft ticks that carry ASFV. In the gene drive, an allele is altered so that it always shows up as the dominant allele in all offspring (not just 50%).

There have been many attempts to develop vaccines for ASFV over several including the intravenous injection of: 1) attenuated viruses (PMID: 31106218, PMID: 29609966, PMID: 27795430, PMID: 28814514, PMID: 33371460, PMID: 32124180, PMID: 31969432, PMID: 33327488, PMID: 34643433), 2) DNA/RNA vaccines, 3) antibody stimulating proteins that are derived from the outer membrane or capsid of the virus, (PMID: 8615037, PMID: 14980493, PMID: 28241999, PMID: 31275307, PMID: 34715492) 4) outer membrane or capsid neutralizing antibodies, (PMID: 28241999, PMID: 33562314), and 5) gene editing (PMID: 29362418, PMID: 34395778, PMID: 32587905).

Live naturally occurring and/or recombinant attenuated virus approaches have been shown to promote robust protection in swine against ASFV (PMID: 31969432), and to date represent the only protective approaches to ASF viral infection in swine. These approaches are meant for temporary relief only against viral infection due to the risk of reversion into more virulent strains of ASFV. Therefore, these technologies are less desirable than more advanced and safer subunit-based vaccines and therapeutics that are currently being sought by multiple laboratories.

DNA/RNA vaccines are highly potent and have been shown to be very effective in protecting hosts from viruses such as SARS-CoV-2. However, few attempts using DNA/RNA vaccine approaches for ASFV infection have been developed due to a lack of targeting and strategy knowledge that is confounded by viral structure and genomic complexities. ASFV is a multi-layered viral particle with at least two mechanisms of infection. Further, the ASFV multi-layered viral particle encompasses a viral genome with more than 150 open reading frames (PMID: 30185597, PMID: 31787524), most that have yet to be characterized.

Current subunit protein vaccine approaches that are designed to stimulate the immune system have fallen short of prolonged protection against the virus for a number of reasons. First, (as above) a lack of knowledge of protein function, genomic properties and viral infection cycles has impaired meaningful translation into a robust vaccine and/or therapeutic. Second, a rapid-fire combination of subunit protein injections that emulate multiple viral antigens has had minimally lasting protective effects likely due to inaccurate timing of treatment (per protein antigen), incorrect concentrations, and over-burdening of the immune system to produce a functional and lasting counter offensive, and most importantly, not considering the viral method of replication (as defined in FIG. 2).

Neutralizing antibody approaches have also been met with limited protective quality against primary infections of ASFV in swine due to the limited knowledge related to the minimal structural, genomic, and replication cycles of the virus. Additional antibody-based therapeutic prophylactic approaches include the use of convalescent serum or selected/engineered monoclonal antibodies. Convalescent serum consisting of protective polyclonal pools of antibodies are likely not strong or stable enough to recognize viral antigen to elicit a prolonged immune response in the swine. Further, engineered, screened, or in silico designed monoclonal antibodies or monoclonal equivalents such as camelid antibodies may likely be better candidates than polyclonal antibodies (pooled either in vitro or derived and expressed in vivo). Additionally, antibody therapeutic approaches must take into account the lysogenic (outer membrane containing virus) and lytic (capsid-based virus) cycles and the timing of treatment (as previously described by Chen and Malcolm in PCT/US20/50939). For example, if the lysogenic outer membrane containing virions are not neutralized and the cycle is allowed to proceed (hidden from antibody therapies) to a lytic stage, the immune (and therapeutic advantage) will be overcome by virus flooding the body of the swine (as in FIG. 1B).

CRISPR gene editing has been shown to ablate the virus and prevent its spread in culture (PMID: 29362418). This powerful technique holds promise to cure ASFV in swine, but such therapies will never make it to market due to their high cost, especially when taking into consideration the low cost of swine per head.

Due to these challenges, there is a need to develop novel strategies for the treatment of ASFV based on newly discovered structural (PMID: 31624094), genomic, replication cycle, and immune interactions with viral antigens (FIGS. 3-5).

Structural Discoveries and Corresponding Inventions.

ASFV is a multi-layered and extremely stable virus. ASFV has 5 layers: 1) a nucleoid, 2) a core shell, 3) an inner membrane 4) a capsid and 5) an outer membrane (FIG. 1A).

ASFV goes through two infectious cycles, a rapid lysogenic cycle followed by an overwhelming lytic cycle (FIGS. 2A-2E). The lysogenic cycle begins through two mechanisms of action
1) As a five-layered virus that contains an outer membrane that infects macrophage through a red blood cell-to-macrophage mediated destruction pathway (FIG. 2A) and 2) as a capsid-based virion (without an outer membrane) that infects macrophage directly via endo/pinocytosis (PMID: 2937495, PMID: 8219802, PMID: 9646445, PMID: 25662020, PMID: 29117102, PMID: 22719252) (FIG. 2B). In the first lysogenic MOA, where ASFV contains an outer membrane, the viral transmembrane proteins CD2v (EP402R) attaches to circulating red blood cells (RBCs) and cause the RBCs to aggregate, as previously observed (PMID: 340610, PMID: 3307125, PMID: 8372447, PMID: 21248037) (FIG. 2C), likely facilitating the entry of the virus into the macrophage cytoplasm through an unknown receptor-mediated mechanism. The hemadsorption method of action of the virus (facilitated by CD2v (EP402R) likely centralizes the concentration of the virus thereby increasing the degree of macrophage infection. (FIG. 2D). Growing evidence is revealing that the ASF virion enters the macrophage via endo/pinocytosis, driven by conformational changes in the CD2v (EP402R) outer membrane protein where its peptide sequences ([KPCPPP]3 are exposed and facilitate viral escape into the cytoplasm (PMID: 34006158, PMID: 27110717). CD2v (EP402R) also inhibits T-cell responses. Another outer membrane viral protein called EP153R forms dimers that bind to MHC Class 1 surface molecules to block antigen presentation to T-cell responses, thereby hiding the infected cell from the immune system (PMID: 21069396) (FIG. 2D). p54 (E183L) is an integral protein that lies in the inner envelope of the virus with peptide tags that protrude from the capsid (PMID: 15047843, PMID: 34834930, PMID: 31787524). Antibodies raised against p54 (E183L) have been shown to have strong neutralizing effects (PMID: 34715492, PMID: 32915442). p54 (E183L) is an early protein of the infectious cycle (lysogenic) that helps to shuttle cytoplasmic ASF virions, via dynein interactions, to ‘virus factory’ regions within the endoplasmic reticulum (ER) (PMID: 15225638) (FIG. 2D). Another candidate outer membrane viral protein, with an N-terminal transmembrane domain and multiple C-terminal glycosylation sites, which has been shown to be critical to the viral replication cycle is I177L. The function of I177L has yet to be fully defined, but its deletion profoundly diminishes the virus' ability to replicate, suggesting an early-stage role in the lysogenic cycle (FIG. 1C).

Once ASFV has entered the macrophage through an unknown mechanism that is likely receptor-mediated (either by EP402R or I177L, or a combined mechanism from either receptor), the virus is released into the cytoplasm, and trafficked to viral factories (via p54 (E183L)/dynein interactions) in the ER where it begins to replicate (PMID: 15047843) (FIG. 2D). The newly formed virions in the cytoplasm then locate to the cytoplasmic membrane of the infected macrophage, where they bud as mature virions into circulation of the swine (FIG. 2D). It is through this budding process where the virus acquires its outer membrane (from the host cell). Once the new outer membrane-containing virion is released from the infected macrophage, it targets new RBCs to begin the process again. As the lysogenic infectious cycle rapidly overtakes the population of macrophage in the swine, there is likely a trigger (such as a late-stage promoter regulated by an increased amount of specific and yet to be defined viral protein PMID: 32075923) that switches the replication cycle from lysogenic (where apoptosis is suppressed) to the lytic cycle while also activating cellular apoptosis pathways (PMID: 12805900) (FIG. 2E).

Once the lytic cycle begins, capsid-based virions explode from the cell and spread quickly through the organism (FIG. 2E) that now has a suppressed T-cell (via viral protein CD2v (EP402R)-mediated suppression) and macrophage response (due to infection and viral protein EP153R-mediated suppression PMID: 21069396) (FIG. 2D). At this time, the amount of virus released into the body overwhelms any pre-existing antibody response (either naturally occurring from B-cells or induced by protein antigen vaccines, or antibody therapeutics) (FIG. 2E). For this reason, simply targeting the capsid antigens (mostly late lytic cycle) for vaccination or therapeutics regimens will not work, and this has likely been the underlying issue with many attempts from countless groups (FIGS. 1B and 2E).

This model, based off recent structural and viral replication data (PMID: 31787524, PMID: 34643433), allows for a strategic plan to be implemented to fulfill the need for meaningful, safe and long-lasting vaccines and/or therapeutics (FIG. 3—‘protein subunit and antibody legend’, and FIG. 4, FIG. 5).

The three-fold strategy depending on the temporal properties of the lysogenic and lytic cycles of ASFV:
(1) By creating antibodies (through either a sub-unit vaccine, or mRNA/DNA vaccine, or direct antibody therapeutic approach) that neutralize; i) the viral outer membrane proteins CD2v (EP402R), and/or EP153R, and/or I177L, and/or p12 (061R), ii) the capsid structural proteins p14.5 (E120R), and/or p72 (B646L) and/or p49 (B438L), iii) the early stage highly antigenic secretory and viral to host cell receptor binding facilitator protein p30 (CP204L) and/or, iv) the late stage highly antigenic inner envelope protein p54 (E183L) that has exposed trans-capsid regions at the onset of infection, both the lysogenic and lytic replication cycle of the virus is prevented.

Specific to blocking of the early lysogenic cycle:
a) The blocking/neutralization of EP402R (by any of the methods mentioned herein), prevents the binding of ASFV to RBCs. By preventing RBC aggregation, the hemadsorption mechanism of proximity for the viruses host cell is vastly diminished, but not fully abrogated. There are many existing cases were hemadsorption by a virus stimulates viral infection by placing the virus in dense regions and proximity to its host cell and attracting multiple types of immune effectors (these include viruses with or without an outer membrane). Further, the blocking/neutralization of EP402R prevents T-cell inhibition by EP402R, allowing an immune response to be mounted, coupled through the re-exposed MHC Class I molecules on macrophage after EP153R competitive blocking.
b) The competitive blocking of EP153R (by any of the methods mentioned herein) for the MHC Class I molecules on macrophage, prevents the virus from inhibiting the activity of MHC Class I molecules on macrophage thereby allowing subsequent immune responses. The competitive inhibition of EP153R allows the infected cell to continue to present antigens to the immune system to mount a response.
c) The blocking of viral entry into the macrophage (by any of the methods mentioned herein) that is likely facilitated by EP402R, I177L, and p30 (CP204L). In one method, the p30 secretory protein binds to the macrophage, creating a cell receptor/viral ligand junction that facilitates or enhances ASFV uptake when it is either bound or unbound to RBCs. EP402R has been shown to contain a membrane crossing peptide (PMID: 34006158). This mechanism likely parallels other fusogen-type acidic dependent mechanisms that occur after the virus has been internalized into a cellular compartment such as an endosome (PMID: 12820183, PMID: 17429580)
d) The neutralization of p12 (061L), an outer membrane and inner envelope viral protein may also prevent the lysogenic and lytic cycle at the different stages of replication (by any of the methods mentioned herein). The function of p12 (061 L) has yet to be determined but has implicated in virus attached to the host cell (PMID: 8219802).

By combining blocking strategies for each of these mechanisms of action, the lysogenic cycle can be halted, the immune system unsuppressed (by recoupling between MHC Class I antigen presentation and T-cell responses), and downstream B-cell mediated neutralization of outer membrane containing virions (if facilitated by antibody therapeutic blocking/neutralizing methods).

Specific to blocking of the early lytic cycle:
a) The neutralization of p72 (B646L) with antibodies has been shown to have temporary ASFV infection neutralizing results. As mentioned above, it is likely the lytic cycle (that follows a vigorous lysogenic replication cycle) overwhelms the swine with viral titers that far exceed any permanent or long-lasting protection effects from antibodies that bind to p72 (B646L) or other capsid proteins—the concentration of the therapy is too low in an overwhelmingly high viral titer environment. Thus, early treatment with neutralizing antibodies (or by any of the methods mentioned herein) in combination with antibodies that block the lysogenic cycles (or by any of the methods mentioned herein), will likely help to prevent early capsid-based virion infection that primes initial lysogenic cycle replication, from progressing to the lytic stage.
b) The neutralization of p49 (B438L), another major structural protein of the viral capsid, with targeted antibodies (or by any of the methods mentioned herein) prior to or during early infection will help to prevent host cell infection by endo/pinocytosis, in a similar manner to p72 neutralizing antibodies.
c) The neutralizing/blocking (by any of the methods mentioned herein) of p14.5 (E120R), a capsid structural protein that becomes functionally activated in the host cell cytoplasm during late the stage of infection (lytic) that suppresses interferon beta production by interacting with IRF3, would restore this immunological function while simultaneously blocking the virus from entering/continuing the lytic stage of replication. p11.5 (A137R) is another capsid structural protein that has downstream immune suppressing functions like p14.5 (E120R). p11.5 (A137R) inhibits the production of type I interferon by binding to TANK-binding kinase 1 (TBK1) (PMID: 35412346)
d) The blocking of p54 (E183L), a highly antigenic inner envelope protein with peptide protrusions that span the capsid, which is expressed in the later stages of ASFV infection (and thus likely a lytic stage structural component), also will likely mount a neutralizing effect against capsid-based virions, thereby preventing capsid-based infection of host cells that occurs through endo/pinocytosis. Further, blocking/neutralization of p54 (E183L) will likely inhibit its viral transport to the ER function, thereby inhibiting both lysogenic and lytic viral replication stages.
e) The blocking of p30 (CP204L) prior to or early during infection may also prevent host receptor-to-viral ligand interactions that may occur through endo/pinocytosis entry pathways.

The blocking/neutralizing methods mentioned herein can be used to target any of the viral proteins associated with the lysogenic and lytic replication cycles to achieve cycle disruption, but at least one target for each cycle must be used simultaneously to effectively achieve that disruption. (FIG. 4 and FIGS. 5A and 5B, and TABLES X).

By inhibiting each of these outer membrane and/or capsid-based proteins in defined combinations (but not limited to these proteins) from interacting with a) RBCs and T-cell receptors, ASFV's primary MOA of entry into macrophage (which has yet to be defined) b) MHC Class I molecules on macrophage (and other antigen presenting cells), c) signal transduction proteins involved the inhibition of anti-viral cytokine expression and d) other receptor-mediated functionalities, the virus is: 1) blocked from other cellular interactions, 2) the T-cell response is no longer inhibited, 3) the macrophage MHC Class 1 complexes continue to be expressed thereby aiding to suppress the early lytic cycle from taking root (FIG. 5D1) and, 4) anti-viral cytokine inhibition will be prevented, respectively. Any combination of CD2v (EP402R), EP153R, p1177L, and p12 (063L), can be used to treat the swine and block the viral lysogenic cycle from taking root (FIG. 4). Each protein and protein subunit in development for a sub-unit vaccine, mRNA/DNA vaccine, or to create/engineer therapeutic antibodies can have at least 50%, 55%, 60%, 65%, 70%, 75%, 80%, 85%, 90%, 95%, 97%, 98%, or 99% sequence identity similarity.

(2) By creating antibodies that neutralize the capsid proteins on the virions that do not contain an outer membrane, early infection through capsid-related endo/pinocytosis can be inhibited. Capsid proteins p14.5 (E120R), p49 (B438L), p72 (B646L), p30 (CP204L), p54 (E183L), and p12 (063R), represent formidable targets for vaccines and/or therapeutic regimen approaches (FIG. 1C). p72 (B646L) and p49 (B438L) are the major structural proteins of the ASFV capsid but multiple attempts to create vaccines using these proteins have been unsuccessful by only offering minimal protection. As mentioned above, the likely reason is that once the lytic cycle is initiated in infected swine, the amount of viral particle far exceeds the neutralizing effects of any antibody therapeutic or B-cell response (FIG. 1B), and the number of macrophages to elicit the destruction of antibody-neutralized virus is vastly diminished. Therefore, targeted antibody responses are necessary to first neutralize the lysogenic ASFV-type virions (those with outer membranes), as well as lytic ASFV-type virions (those with no outer membrane and consisting of exposed capsid antigen targets), and those that are secretory and facilitate host cell binding (FIG. 4). Each protein and protein subunit in development for a sub-unit vaccine, mRNA/DNA vaccine, or to create/engineer therapeutic antibodies can have at least 50%, 55%, 60%, 65%, 70%, 75%, 80%, 85%, 90%, 95%, 97%, 98%, or 99% sequence identity similarity.

(3) Additional protection can be implemented by engineering each of the targeted antibodies (toward CD2v (EP402R), EP153R, p72 (B646L), p54 (E183L), p30 (CP204L), and p12 (061R), p49 (B438L), and I177L, but not limited to these proteins) with a swine equivalent of human CD47 tag (FIG. 7 and FIG. 8— RNA and partial protein sequence, respectively) constructed into the Fc region of the antibodies (FIG. 6). CD47 is a ‘don't eat me’ 5 transmembrane receptor protein that is naturally occurring on cells (PMID: 29709247, U.S. Pat. Nos. 9,050,269, 8,377,448, USPN 8,0064,306). In humans, CD47 receptors bind to SIRPα forming a signaling axis that triggers the prevention of programmed cell removal PCR) (PMID: 11509594). This prevents macrophage from devouring cells that belong to the organism. However, CD47 has been reported to play the opposite role regarding macropinocytosis. CD47 containing exosomes have been shown to trigger macropinocytosis through its interaction with TSP1 on the surface of monocytes and macrophages. This interaction triggers the upregulation of Nox1 leading to membrane ruffling and initiation of macropinocytosis (PMID: 27958762). Thus, although the CD47 isoform 2 extracellular domain fused to the Fc region of an antibody will likely prevent phagocytosis of the neutralized virus, the virus (covered with engineered antibody) may enhance its uptake via macropinocytosis. To get around this challenge, the CD47 isoform extracellular domain will be engineered/selected to prevent its interaction with TSP1, while retaining its interactive properties with SIRP-α. In this scenario, both phagocytosis and macropinocytosis are inhibited by the mutantCD47 (mCD47) isoform 2 extracellular domain (FIG. 6). By integrating the extracellular domain of mCD47 (isoform 2) into the Fc region of the targeted antibodies, the uptake of virus by macrophage can be eliminated by:
a) Prevention of neutralized virus phagocytic events.
b) Prevention of macropinocytosis that has been predicted to occur with capsid based ASFV (PMID: 22719252, PMID: 29117102).
c) Prevention of endocytosis indirectly. Although there is no evidence that CD47 regulates endocytosis, a combination of targeted antibody/protein vaccine therapy would diminish the capabilities of the ASFV infectious cycles considerably. It is likely endocytosis of ASFV becomes the predominant form of infection after the lytic cycle has taken over, and the remaining macrophage (and other cells) uptake the virus when it is at higher concentrations. By implementing this targeting strategy and incorporating the CD47 or mCD47 isoform 2 extracellular domain signals into the Fc region of each targeted antibody, the lytic cycle will likely never take root.
d) The mCD47 tagged (through antibody binding) ASF virus would then be cleared through the neutrophil pathway. Neutrophils have not been reported to be infected by ASFV (FIG. 6).

Strong antibody responses can be triggered in a number of ways. Therapeutics and Vaccines:

Therapeutic. The antibodies (for each desired target) can be selected and engineered using a platform screening approach or designed using artificial intelligence software such as Alphafold or Rosetta. Once the very strong, robust, highly selective, precise, specific, high affinity and high avidity antibodies are selected and/or A.I. designed, these antibodies (monoclonal) can be used as a therapeutic for direct injection, post biochemical and immunological pre-clinical testing. The therapeutic can be used to treat infected swine or as an antibody vaccine to protect swine against infection—prophylactic.

Therapeutic. Further, the epitope sequences can be derived from the monoclonal antibody selection process. These sequences can be used to engineer an antibody that contains the serotype-2 CD47 (mCD47) extracellular domain fused to the Fc region of the antibody. These engineered antibodies (against any desired target) can be used as a therapeutic to neutralize ASFV and prevent infection into macrophage.

Vaccine. The antibodies can be naturally stimulated by injecting the proteins of each target into the swine model. The protein antigen subunit vaccine will then stimulate the B-cells to create antibodies against them, and therefore the virus. Variants may include any peptide derivation from the protein targets with differences up to 90%). The antibodies produced will depend on the concentration of proteins injected and adjuvant release for prolonged effects.

Vaccine. The proteins (in any combination or concentration/dose—toward CD2v (EP402R), EP153R, p72 (B646L), p54 (E183L), p30 (CP204L), and p12 (061R), p49 (B438L), and I177L, (but not limited to these proteins account for critical targets that fall within the lysogenic/lytic dual treatment model. Each protein or subunit peptide related to the proteins can be expressed by delivering RNA/DNA transcripts in targeted liponanoparticles, exosomes, nanovesicles, biomimetic exosomes, AAVs, Anellovirus or Clews to B-cells to produce the protein antigens and elicit a more robust and lasting antibody effect. This approach will induce naturally structured (without a CD47 Fc region tag) antibodies. The delivery vehicle can also be targeted to B-cells using ligands that recognize CD19 receptors on B-cells (for example), but not limited to the CD19 target.

The receptor for ASFV on macrophage is unknown. Endo/pinocytosis has been shown to be one method of entry for ASFV. Due to the inefficient process of this method of infection, it is likely to occur in very early infection to start lysogenic replication processes and become more prominent during the lytic replication stage when ASFV virions are in very high concentrations. A two-hybrid system can be used to determine the viral ligand (bait) to cellular receptor (prey) interaction to define this MOA. The viral proteins p12 (061R), p54 (E183L), p49 (B438L), EP153R, EP402R (potential dual function, infection into host cell cytoplasm due to the presence of cell membrane penetrating peptides sequences and RBC binding), and I177L each have been predicted as potential viral ligands for cellular receptor-mediated infection.

p12 (061L) has been shown to exist in the outer membrane (lysogenic) and between the inner membrane and capsid (lytic) (PMID: 8219802, PMID: 9213394).

p54 (E183L) is an inner envelope protein component with a capsid exposed peptide segment. Antibodies raised against p54 (E183L) have been shown to slow the infection of the ASFV but remain insufficient to prevent infection (PMID: 14980493).

p49 (B438L) exists between the inner membrane and the capsid (lytic) and has a predicted receptor domain (PMID: 31624094).

EP153R, an outer membrane protein reduces the expression of MHC Class 1 surface molecules, suggesting it has role in direct macrophage contact at the cellular surface and a possible MOA for viral entry into the cell (PMID: 29609966, PMID: 21069396).

I177L has been predicted to be an outer membrane (lysogenic) and inner membrane (lytic) protein, containing a transmembrane domain. Its deletion significantly reduces infection (PMID: 33925435).

EP402R is an outer membrane protein that has been shown to facilitate hemadsorption between the outer membrane containing ASFV virion and RBCs. EP402R has also been shown to contain cell penetrating peptides.

By defining the viral ligand and cellular receptor interacting proteins and interaction MOA, the receptor can be blocked with small molecules, or antibodies, or nanobodies, or mutated virus that competes for the receptor, or nucleic acid competitors/binders.

Further, GMO swine or engineered macrophage (for replacement/substitution therapy) can be engineered to have receptors that have been altered in a manner to prevent the binding of ASFV. Similar approaches have been attempted to engineer human cells to be resistant to viruses such SARS-CoV-2 (ACE receptors) and HIV (CCRS delta mutations).

However, methods of treating the virus itself are still needed.

To date, the cellular receptor(s) for ASFV has not been identified, but there is evidence that the virus enters through a dynamin-dependent and clathrin-mediated endo/pinocytosis process in monocyte or macrophage cells (PMID: 29978065). Attempts to create strong antibody responses against viral antigens of ASFV have been met with poor results.

Gene editing allows DNA or RNA to be inserted, deleted, or replaced in an organism's genome by the use of nucleases. There are several types of nucleases currently used, including meganucleases, zinc finger nucleases, transcription activator-like effector-based nucleases (TALENs), and clustered regularly interspaced short palindromic repeats (CRISPR)-Cas nucleases. These nucleases can create site-specific double (or single) strand breaks of the DNA in order to edit the DNA. Targeting the genome of receptors requires precise cuts to the viral genome and no off-target effects that could be harmful to the subject.

US Patent Application Publication No. 2014/0357530 discloses compositions, methods applications and screens used in functional genomics that focus on gene function in a cell and that use vector systems and other aspects related to Clustered Regularly Interspaced Short Palindromic Repeats (CRISPR)-Cas systems and components thereof. The application discloses modification of short portions of DNA, creating a 5′ overhang that is at most 200 base pairs, preferably at most 100 base pairs, or more preferably at most 50 base pairs.

U.S. Pat. No. 10,266,850 discloses DNA-targeting RNA that comprises a targeting sequence and, together with a modifying polypeptide, provides for site specific modification of a target DNA and/or a polypeptide associated with the target DNA. Also disclosed are methods of modulating transcription of a target nucleic acid in a target cell, generally involving contacting the target nucleic acid with an enzymatically inactive Cas9 polypeptide and a DNA-targeting RNA.

Gene editing has also been used to create point mutations. PMID: 30323312 teaches base editing, a newer genome-editing approach that uses components from CRISPR systems together with other enzymes to directly install point mutations into cellular DNA or RNA without making double-stranded DNA breaks. DNA base editors comprise a catalytically disabled nuclease fused to a nucleobase deaminase enzyme and, in some cases, a DNA glycosylase inhibitor. RNA base editors achieve analogous changes using components that target RNA. Base editors directly convert one base or base pair into another, enabling the efficient installation of point mutations in non-dividing cells without generating excess undesired editing byproducts.

Cas/deaminase fusion proteins have also been used to make point mutations. PMID: 30271918 used a nickase Cas9-cytidine deaminase fusion protein to direct the conversion of cytosine to thymine within prokaryotic cells, resulting in high mutagenesis frequencies in Escherichia coli and Brucella melitensis. US Patent Application Publication No. 20160304846 also discloses fusion proteins of Cas9 and nucleic acid editing enzymes or enzyme domains, e.g., deaminase domains, for editing a single site within the genome of a cell or subject.

There remains a need for treating and preventing viral infections (such as ASFV) that undergo a combination of early lysogenic viral replication, followed by late-stage lytic viral replication by interrupting their interactions with cellular receptors and cellular internalizing pathways (such as phagocytosis, macropinocytosis and endocytosis).

### SUMMARY OF THE INVENTION

The present invention provides for a method of preventing and treating viral infections in animals (and preferably ASFV in porcine). Specifically, the invention couples the inhibition/blocking/neutralization of viral proteins associated with the lysogenic stage of replication, to the inhibition/blocking/neutralization of viral proteins associated with the lytic stage of replication in a simultaneous manner to disrupt and prevent both cycles from occurring. The result is the prevention of viral infection.

In more detail, the present invention targets viral ligand interactions associated with cellular receptors involved in hemadsorption, receptor-mediated viral infection of host cells, the inhibition of immune cell suppression, and the inhibition of anti-viral cytokine suppression that are integral with virions produced from a lysogenic replication cycle containing an outer membrane, and by simultaneously neutralizing structural capsid proteins in virions produced from a lytic replication cycle that lack an outer membrane.

TABLE 1
Treatment can be accomplished using the following proteins that are defined
in either the lysogenic or lytic viral cycle and in various combinations
to effectively achieve viral replication and infectious disruption

| | | | | | Lysogenic | Lytic |
|---|---|---|---|---|---|---|
| Protein | Location | Cycle | Temporality | Function | Disruption | Disruption |
| CD2v | OM | Lyso | Early | Hemadsorption/ | ✓ | |
| (EP402R) | | | | T-Cell Inhibition | | |
| EP153R | OM | Lyso | Early | MHC I Inhibition | ✓ | |
| I177L | OM | Lyso | Early | Receptor | ✓ | |
| | | | | Associated | | |
| p12 (O61R) | OM/IEV | Lyso/ | Early/Late | Receptor | ✓ | ✓ |
| | | Lyt | | Associated | | |
| p14.5 | CA | Lyso/ | Early | Interferon Beta | ✓ | ✓ |
| (E120R) | | Lyt | | Inhibition | | |
| P11.5 | CA | Lyso/ | Early | Type I Interferon | ✓ | ✓ |
| (A137R) | | Lyt | | Inhibition | | |
| p72 (B646L) | CA | Lyt | Early/Late | Structural | | ✓ |
| p49 (B438L) | CA | Lyt | Early/Late | Structural | | ✓ |
| p30 (CP204L) | Secretory/ | Lyso/ | Early | Receptor Mediator | ✓ | ✓ |
| | CA | Lyt | | | | |
| p54 (E183L) | IEV/CA | Lyso/ | Late | Structural and | ✓ | ✓ |
| | | Lyt | | Viral Shuttling ER | | |

*OM = Outer membrane of virus
*CA = Capsid
*IEV = Inner Envelope

TABLE 2
Examples of target treatment combinations to achieve lysogenic
and lytic viral replication cycle disruption

| | Lysogenic/ |
|---|---|
| Examples of Targets for Treatment | Lytic |
| Combinations (not limited to) | Disruption |
| EP402R + EP153R + B646 | ✓ |
| EP402R + I177L + B646L | ✓ |
| EP402R + EP153R + B438L | ✓ |
| EP402R + I177L + B438L | ✓ |
| EP402R + EP153R + E120R/A137R | ✓ |
| EP402R + I177L + E120R/A137R | ✓ |
| EP402R + O61R + B646L | ✓ |
| EP402R + O61R + B438L | ✓ |
| EP402R + O61R + E120R/A137R | ✓ |
| EP153R + I177L + B646L | ✓ |
| EP153R + O61R + B646L | ✓ |
| EP153R + I177L + B438L | ✓ |
| EP153R + O61R + B438L | ✓ |
| EP153R + I177L + E120R/A137R | ✓ |
| EP153R + O61R + E120R/A137R | ✓ |
| EP402R + EP153R + B646 +/− CP204L or E183L | ✓ |
| EP402R + I177L + B646L +/− CP204L or E183L | ✓ |
| EP402R + EP153R + B438L +/− CP204L or E183L | ✓ |
| EP402R + I177L + B438L +/− CP204L or E183L | ✓ |
| EP402R + EP153R + E120R/A137R +/− CP204L or E183L | ✓ |
| EP402R + I177L + E120R/A137R +/− CP204L or E183L | ✓ |
| EP402R + O61R + B646L +/− CP204L or E183L | ✓ |
| EP402R + O61R + B438L +/− CP204L or E183L | ✓ |
| EP402R + O61R + E120R/A137R +/− CP204L or E183L | ✓ |
| EP153R + I177L + B646L +/− CP204L or E183L | ✓ |
| EP153R + O61R + B646L +/− CP204L or E183L | ✓ |
| EP153R + I177L + B438L +/− CP204L or E183L | ✓ |
| EP153R + O61R + B438L +/− CP204L or E183L | ✓ |
| EP153R + I177L + E120R/A137R +/− CP204L or E183L | ✓ |
| EP153R + O61R + E120R/A137R +/− CP204L or E183L | ✓ |

Treatment can be accomplished through either 1) the (non-) or competitive inhibition of the viral ligand-cellular receptor interactions through engineered antibody therapeutics, 2) virus neutralization by engineered antibody therapeutics, 3) virus neutralization by engineered antibody therapeutic that also prevent phagocytosis and macropinocytosis (CD47/mCD47 domain included in the Fc region of the antibody), 4) virus neutralization by engineered antibody therapeutics with bispecific heavy and light chain epitopes, 5) virus neutralization by engineered antibody therapeutics with bispecific heavy and light chain epitopes that also prevent phagocytosis and macropinocytosis (CD47/mCD47 domain included in the Fc region of the antibody), 6) the (non-) or competitive inhibition of the viral ligand-cellular receptor interactions with small molecules, or 7) cellular receptor altering through gene editing methods, so that the viral entry proteins no longer recognize the natural/wildtype receptor.

Prevention (vaccine) can be accomplished through either 1) immune stimulation (B-cell) through the injection of viral proteins (or domains of the proteins) that are involved with ligand-cellular receptor interactions, 2) immune stimulation (T-cell) through the injection of viral T-cell antigens, 3) immune stimulation (B-cell and T-cell simultaneously) through the injection of viral proteins (or domains of the proteins) that are involved in the ligand-cellular receptor interaction or T-cell antigens, respectively, 4) the delivery (via exosomes, biomimetic exosomes, nanoparticles, AAV, Anellovirus, clews, liposomes) of mRNA encoding viral proteins or domains of the proteins that are involved in ligand-cellular receptor interactions such as to elicit an immune response from B-cells to produce neutralizing antibodies or any one of the combinations above that can be used in a pre-infective/prophylactic manner.

The present invention provides for a method of treating a viral infection in an individual with a virus that is both lysogenic and lytic, by administering a viral antigen that targets protein on an outer membrane of a lysogenic phase of the virus, administering a viral antigen that targets protein on a capsid of a lytic phase of the virus, and treating the viral infection.

The present invention also provides for a composition for treating a viral infection in an individual with a virus that is both lysogenic and lytic including a viral antigen that targets protein on an outer membrane of a lysogenic phase of the virus and a viral antigen that targets protein on a capsid of a lytic phase of the virus.

The present invention also provides for a vaccine for preventing viral infection, including whole and/or partial domains of proteins of both a lysogenic and lytic phase of a virus.

### DESCRIPTION OF THE DRAWINGS

Other advantages of the present invention are readily appreciated as the same becomes better understood by reference to the following detailed description when considered in connection with the accompanying drawings wherein:

FIGS. 1A-1C show the lysogenic and lytic structures of ASFV, FIG. 1A shows ASFV structure, lysogenic (left) vs. Lytic (right), FIG. 1B shows antibodies directed toward capsid proteins do not penetrate virions with outer membranes that are derived from the lysogenic replication cycle, capsid-based neutralizing antibodies are not enough to eliminate all virus, and FIG. 1C shows outer membrane protein targets (top) vs. Capsid protein targets (bottom);

FIGS. 2A-2E are schematics showing the ASFV Infectious and Replication Cycle, FIG. 2A shows outer membrane containing virion infection of swine, the outer membrane virion causes the aggregation of RBCs in circulating blood through the viral proteins CD2v (EP402R), and EP153R, FIG. 2B shows capsid containing virion (no outer membrane) infection of swine, the capsid-based virus infects circulating macrophage and/or monocytes via pinocytosis and/or endocytosis, once in the cell, the virus is shuttled to virus factory regions by p54 (E183L) to begin the lysogenic cycle, the progeny virions (unlike the parent capsid-based virion) are bud from the cytoplasmic membrane of the cell and now contain an outer membrane, the outer membrane containing virions are bud from the cell into the blood, FIG. 2C shows that similar to FIG. 2A, the virions cause RBC aggregation leading to infection of macrophage (mechanism unknown, but likely receptor-mediated) of virion-aggregated RBCs, FIG. 2D shows subsequent infection of macrophage once the virions are internalized possibly through cellular compartmental escape by the activation of EP402R cell penetrating ([KPCPPP]3 peptide. This peptide has been shown to be activated during endo/pinocytosis (PMID: 34006158). Further, as the concentrations of CD2v (EP402R) and EP153R increase (via virion production), they interact with T-cells and the MHC complexes of the macrophage respectively, resulting in the down regulation of T-cell responses and MHC complex mediated immune interactions, FIG. 2E shows as the lysogenic cycle overwhelms the macrophage and increases the number of virions in circulation, genetic switches occur that cause the virus to enter the lytic stage of replication, here, the cells burst open and exponentially increase the amount of infective capsid-based virions into the blood, the capsid-based virions, now at very high concentrations, can infect multiple cell types through the endo/pinocytosis pathways. The runaway infection overwhelms an already strained and suppressed immune system, leading to the animal's death;

FIG. 3 shows an antibody legend and description of Methods of Action (MOAs) for each therapeutic, in relation to the lytic and lysogenic cycles;

FIG. 4 shows alternate protective treatment strategies (vaccines vs. therapeutic);

FIGS. 5A-5D2 are schematics showing proposed vaccine and therapeutic approach to treat ASFV, by attacking the lysogenic and lytic viral cycles with antibodies (either injected prophylactically/therapeutically or stimulated internally by the injection of protein vaccine subunits, ASFV's replication cycle can be blocked, and the virus neutralized, FIG. 5A shows α-CD2v (EP402R) prevents RBC aggregation by blocking CD2v (EP402R) on the outer membrane of the virions, FIG. 5B shows α-EP153R prevents RBC aggregation and virion-mediated MHC blocking, by neutralizing EP153R on the outer membrane of the virion, both 5A and 5B can happen simultaneously on the same virion, FIG. 5B1 shows a virus without the ability to replicate, FIG. 5C shows α-p54 (E183L) prevents viral replication by blocking indirectly the dynein-mediated transport of the internalized virions to viral factories in the ER, and FIGS. 5D1 and 5D2 show α-p72 (B646L) (and other capsid proteins like p30 (CP204L), p14.5 (E120R) and p49 (B438L) neutralizes capsid-based virions in early infection to prevent lytic take over, before it takes root and overwhelms the system;

FIG. 6 is a schematic showing CD47/mCD47 tagging of the Fc region of any of the antibodies mentioned above, serves to neutralize virion antigens while preventing macrophage uptake and potential unintended infection through phagocytic pathways (via the CD2v (EP402R) cell penetrating ([KPCPPP]3 peptide activation) and endo/pinocytosis (mCD47), the neutralized virions are alternately degraded via neutrophil-mediated degradation;

FIG. 7 shows swine CD47 Isoform 2 mRNA sequence; and

FIG. 8 shows swine CD47 Isoform 2 partial protein sequence.

### DETAILED DESCRIPTION OF THE INVENTION

The present invention provides for a method of preventing and treating viral infections (and preferably ASFV in porcine), by simultaneously using disruptive combinations of therapeutics by, i) inhibiting the viral entry protein-to-cellular receptor interaction, ii) inhibiting the suppression of immune responses caused by viral proteins, iii) prevention of proximity mechanisms of infection by disrupting hemadsorption caused by viral proteins and, iv) by neutralizing structural proteins to mount an immune response against the virus. Treatment can be accomplished through either 1) the (non-) or competitive inhibition of the viral ligand-cellular receptor interactions through engineered antibody therapeutics, 2) virus neutralization by engineered antibody therapeutics, 3) virus neutralization by engineered antibody therapeutic that also prevent phagocytosis and macropinocytosis (CD47/mCD47 domain included in the Fc region of the antibody), 4) virus neutralization by engineered antibody therapeutics with bispecific heavy and light chain epitopes, 5) virus neutralization by engineered antibody therapeutics with bispecific heavy and light chain epitopes that also prevent phagocytosis and macropinocytosis (CD47/mCD47 domain included in the Fc region of the antibody), 6) the (non-) or competitive inhibition of the viral ligand-cellular receptor interactions with small molecules, or 7) cellular receptor altering through gene editing methods, so that the viral entry proteins no longer recognize the natural/wildtype receptor.

Prevention (vaccine) can be accomplished through either 1) immune stimulation (B-cell) through the injection of viral proteins (or domains of the proteins) that are involved with ligand-cellular receptor interactions, 2) immune stimulation (T-cell) through the injection of viral T-cell antigens (ref), 3) immune stimulation (B-cell and T-cell simultaneously) through the injection of viral proteins (or domains of the proteins) that are involved in the ligand-cellular receptor interaction or T-cell antigens, respectively, 4) the delivery (via exosomes, biomimetic exosomes, nanoparticles, AAV, Anellovirus, clews, liposomes, or any other suitable delivery methods) of mRNA encoding viral proteins or domains of the proteins that are involved in ligand-cellular receptor interactions such as to elicit an immune response from B-cells to produce neutralizing antibodies or anyone of the combinations above that can be used in a pre-infective/prophylactic manner.

“Animal” as used herein refers to any non-human species of animal.

“Porcine” or “swine” as used herein, can be a domestic pig, wild boar, warthog, or bush pig.

The term “vector” includes cloning and expression vectors, as well as viral vectors and integrating vectors. An “expression vector” is a vector that includes a regulatory region. Vectors are also further described below.

The term “antibody” as used herein refers to a blood protein produced in response to and counteracting a specific antigen. Antibodies combine chemically with substances which the body recognizes as alien, such as bacteria, viruses, and foreign substances in the blood.

The term “mRNA” as used herein refers to a type of RNA in cells that carries genetic information required to make proteins.

The terms “CD47 and/or CD47 domain and/or CD47 extra cellular domain” as used herein refer to a transmembrane protein that is present on many different cell types in all tissues. It is involved in cellular processes such as apoptosis, proliferation, adhesion, and migration.

The terms “mCD47 and/or mCD47 domain and/or mCD47 extra cellular domain” as used herein refer to as a modification of the wild type CD47 transmembrane protein that is present on many different cell types in all tissues. This modification/mutant retains the interaction property with SIRP-α receptors to prevent phagocytosis, but no longer binds to TSP1 thereby interrupting micropinocytosis-mediated viral entry.

The term “gRNA” as used herein refers to guide RNA. The gRNAs in the CRISPR Cas9 systems and other CRISPR nucleases herein are used for altering or editing receptors or genes encoding receptors. The gRNA can be a sequence complimentary to a coding or a non-coding sequence and can be tailored to the particular receptor or gene to be targeted. The gRNA can be a sequence complementary to a protein coding sequence, for example, a sequence encoding one or more viral structural proteins, (e.g., in ASFV the CP2475 gene encodes polypeptide 220 which is cut into the proteins p150, p37, p14, and p34). The gRNA sequence can be a sense or anti-sense sequence. It should be understood that when a gene editing composition is administered herein, preferably this includes one or more gRNA.

“Nucleic acid” as used herein, refers to both RNA and DNA, including cDNA, genomic DNA, synthetic DNA, and DNA (or RNA) containing nucleic acid analogs, any of which may encode a polypeptide of the invention and all of which are encompassed by the invention. Polynucleotides can have essentially any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA) and portions thereof, transfer RNA, ribosomal RNA, siRNA, micro-RNA, short hairpin RNA (shRNA), interfering RNA (RNAi), ribozymes, cDNA, recombinant polynucleotides, branched polynucleotides, plasmids, vectors, isolated DNA of any sequence, isolated RNA of any sequence, nucleic acid probes, and primers, as well as nucleic acid analogs. In the context of the present invention, nucleic acids can encode a fragment of a naturally occurring Cas9 or a biologically active variant thereof and at least two gRNAs where in the gRNAs are complementary to a sequence in a receptor or gene encoding a receptor.

An “isolated” nucleic acid can be, for example, a naturally occurring DNA molecule or a fragment thereof, provided that at least one of the nucleic acid sequences normally found immediately flanking that DNA molecule in a naturally occurring genome is removed or absent. Thus, an isolated nucleic acid includes, without limitation, a DNA molecule that exists as a separate molecule, independent of other sequences (e.g., a chemically synthesized nucleic acid, or a cDNA or genomic DNA fragment produced by the polymerase chain reaction (PCR) or restriction endonuclease treatment). An isolated nucleic acid also refers to a DNA molecule that is incorporated into a vector, an autonomously replicating plasmid, a virus, or into the genomic DNA of a prokaryote or eukaryote. In addition, an isolated nucleic acid can include an engineered nucleic acid such as a DNA molecule that is part of a hybrid or fusion nucleic acid. A nucleic acid existing among many (e.g., dozens, or hundreds to millions) of other nucleic acids within, for example, cDNA libraries or genomic libraries, or gel slices containing a genomic DNA restriction digest, is not an isolated nucleic acid.

Isolated nucleic acid molecules can be produced by standard techniques. For example, polymerase chain reaction (PCR) techniques can be used to obtain an isolated nucleic acid containing a nucleotide sequence described herein, including nucleotide sequences encoding a polypeptide described herein. PCR can be used to amplify specific sequences from DNA as well as RNA, including sequences from total genomic DNA or total cellular RNA. Various PCR methods are described in, for example, PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies are also available by which site-specific nucleotide sequence modifications can be introduced into a template nucleic acid.

Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >50-100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. The isolated nucleic acids of the invention also can be obtained by mutagenesis of, e.g., a naturally occurring portion of a Cas9-encoding DNA (in accordance with, for example, the formula above).

In the methods of the present invention, many different viruses can be treated or prevented in animals, especially porcine. Most preferably, the virus is ASFV. Other animal viruses can include Pseudorabies virus, Bluetongue virus, Foot-and-mouth disease virus (serotypes A, 0, C, SAT1,SAT2, SAT3, Asia1), Japanese encephalitis virus, Rabies virus, Rift Valley fever virus, Rinderpest virus, Vesicular stomatitis virus, West Nile fever virus, BSE prion, Bovine viral diarrhea virus, Bovine leukemia virus, Bovine herpesvirus 1, Lumpy skin disease virus, Caprine arthritis and encephalitis virus, Peste-des-petits-ruminants virus, Scrapie prion, sheeppox and goatpox viruses, African horse sickness virus, Eastern equine encephalomyelitis virus, Western equine encephalomyelitis virus, Equine infectious anemia virus, Equine influenza virus, Equine herpesvirus 4, Equine arteritis virus, Venezuelan equine encephalomyelitis virus, Classical swine fever virus, Nipah virus, Porcine reproductive and respiratory syndrome virus, Swine vesicular disease virus, Transmissible gastroenteritis virus of swine, Avian infectious bronchitis virus, Infectious laryngotracheitis virus, Duck hepatitis virus, High and low pathogenic avian influenza viruses, Infectious bursal disease virus, Marek's disease virus, Newcastle disease virus, or Avian metapneumovirus. The virus can also be generally of the type papovaviruses, simian virus-40, adenoviruses, herpesviruses, pox viruses, picornaviruses, togaviruses, rabies viruses, influenza viruses, or reoviruses.

In performing the methods of the present invention, first, receptor screening is performed. A discovery platform is utilized (yeast two hybrid-based or biochemical interaction assays) for the identification of the cellular receptors that interact with one or more (or in any combination thereof) of the viral attachment and entry proteins/ligands such as CD2v (EP402R), I177L, p12 (061R), p30 (CP204L) receptor mediator, but not limited to these viral protein ligands.

There are multiple yeast two hybrid, mammalian two hybrid, and phage display approaches that can be used for this purpose. Luo, et al. (PMID: 9043710) describes a mammalian two-hybrid system. One protein of interest is expressed as a fusion to the Gal4 DNA-binding domain and another protein is expressed as a fusion to the activation domain of the VP16 protein of the herpes simplex virus. The vectors that express these fusion proteins are cotransfected with a reporter chloramphenicol acetyltransferase (CAT) vector into a mammalian cell line. The reporter plasmid contains a CAT gene under the control of five consensus Gal4 binding sites. If the two fusion proteins interact, there will be a significant increase in expression of the cat reporter gene. Fields, et al. (PMID: 2547163) describes a yeast two-hybrid system with a GAL4 DNA-binding domain fused to a protein X′ and a GAL4 activating region fused to a protein Y. If X and Y can form a protein-protein complex and reconstitute proximity of the GAL4 domains, transcription of a gene regulated by UASG occurs. Smith (Science. 1985 Jun. 14; 228(4705):1315-7) describes a phage two-hybrid system wherein foreign DNA fragments can be inserted into filamentous phage gene III to create a fusion protein with the foreign sequence in the middle. The fusion protein is incorporated into the virion, which retains infectivity and displays the foreign amino acids in immunologically accessible form. These “fusion phage” can be enriched more than 1000-fold over ordinary phage by affinity for antibody directed against the foreign sequence.

The receptor screening can be performed generally as follows. A library of swine/porcine genes is expressed in yeast or phage (phage can be used to screen far more). The expressed proteins then decorate the outside of the yeast cell/phage. An HPLC column can be made of the ASFV Capsid or of proteins or other potential ligands. The yeast cells or phage are incubated with the immobilized ASFV receptor ligand of choice. The cells or phage are washed, collected, and repeated to enrich. The sample is collected, and the receptor identified using typical biochemical/genetic methods defined by each hybrid/phage system.

The receptor and viral ligand interaction can be either competitive inhibition or noncompetitive inhibition. Competitive inhibition occurs when a chemical substance, small peptide, or antibody inhibits the effect of another by competing with it for binding, i.e., it resembles the normal substrate that binds to the receptor. Non-competitive inhibition occurs when the inhibitor reduces activity of the receptor and binds equally well to the receptor whether or not it has already bound the substrate.

A small molecule inhibition treatment can be derived upon the discovery of receptor. Once the interaction between viral ligand and cellular receptor is defined, small molecule disruptive screens (protein-protein interaction/disruption via two hybrid systems or others) is utilized to define small molecule candidates that can inhibit the interaction. Variations of the two hybrid systems can be used, for example a repressed transactivator (RTA) screen. In this screen, the small molecule library is added to yeast that only grows on selective media when the swine receptor peptide and the viral receptor/ligand peptide are locked in an interaction. By adding the small molecule library, one looks for those that disrupt the interaction. Once identified, which small molecule is the most robust, safe, and efficacious can be determined. Hirst, et al. (PMID: 11447261) describes a repressed transactivator (RTA) system employing the N-terminal repression domain of the yeast general repressor TUP1. TUP1-GAL80 fusion proteins, when co-expressed with GAL4, are shown to inhibit transcription of GAL4-dependent reporter genes. Joshi, et al. (PMID: 17515203) has used this system in screening for inhibitors of protein interactions from small molecule compound libraries. The libraries used for screening and testing for the present invention can come from the sea, rainforest, or be synthetic. Peptide and antibody libraries can also be used. Further screening and testing can be conducted to narrow the number of small molecules and test for the safety and efficacy in cell culture and animal models.

A genetically modified cellular receptor can be used for prevention of the virus binding through dysfunction or other disruption of entry proteins. Once the cellular receptor is identified, specifically, the amino acids within the receptor that are critical for the recognition of the viral protein ligands, gene editing tools (such as, but not limited to, CRISPR, ZFNs, TALENs, further described below) can be used to alter (by substitution or deletion) the receptor encoding gene(s) with non-disruptive (functionally retainable protein) amino acid sequence(s) that block viral entry. The entry proteins are otherwise structural or functional membrane proteins. Their alteration can be at the genetic level affected by gene editing, but their natural function may need to be preserved so as to not disrupt or otherwise kill the target cells.

If glycosylation is needed for the receptor, swine macrophage cellular extracts can be added in the yeast/phage expressed libraries to force the glycosylation of the surface expressed peptide on the yeast/phage.

In an alternative to the above method, the viral protein can be isolated on a column as described above, then swine/porcine isolated macrophage/monocyte cells can be run over the column, incubated, then the cells can be enriched by elution (keeping the interaction intact). Once the isolated macrophage/monocyte is interacting with the viral receptor/ligand isolated, an antibody that recognizes the viral ligand can be added and then the synapse can be observed under a microscope. The single cell can be isolated and then the cellular receptor identified.

This gene editing approach can be conducted in swine embryonic lineages to create a genetically modified swine organism that is resistant to ASFV infection.

The gene editors used in the present invention can include any of the gene editors listed below. Any method of action can be used, including endonuclease cutting of DNA or RNA, guided by gRNAs. The nucleases work by cutting out or altering at the base pair level, the endogenous swine receptor sequences and replacing them using HDR with methods like HITI (non-dividing embryonic cells) or traditional HDR in dividing embryonic cells with one or more gRNAs. Gene editing can be used to create point mutations or multiple mutations that result in desired receptor. Cas/deaminase fusion proteins can be used to make point mutations.

Gene replacement can also be performed, which requires excision of a gene followed by replacement of the gene with a new gene that has an altered sequence that expresses a mutant (yet functional) receptor that blocks viral entry. Gene editing can be used to replace a wild type gene with an engineered gene that contains the mutant sequences allowing for the expression of the replacement receptor. Once the gene is excised, it can be replaced using gene replacement approaches (homology-directed recombination) in either dividing or non-dividing cells.

Zinc finger nuclease (ZFN) creates double-strand breaks at specific DNA locations. A ZFN has two functional domains, a DNA-binding domain that recognizes a 6 bp DNA sequence, and a DNA-cleaving domain of the nuclease Fok I.

TALENs (transcription activator-like effector nucleases) include a TAL effector DNA-binding domain fused to a DNA cleavage domain that creates double strand breaks in DNA.

Human WRN is a RecQ helicase encoded by the Werner syndrome gene. It is implicated in genome maintenance, including replication, recombination, excision repair and DNA damage response. These genetic processes and expression of WRN are concomitantly upregulated in many types of cancers. Therefore, it has been proposed that targeted destruction of this helicase could be useful for elimination of cancer cells. Reports have applied the external guide sequence (EGS) approach in directing an RNase P RNA to efficiently cleave the WRN mRNA in cultured human cell lines, thus abolishing translation and activity of this distinctive 3′-5′ DNA helicase-nuclease.

The Class 2 type VI-A CRISPR/Cas effector “C2c2” demonstrates an RNA-guided RNase function. C2c2 from the bacterium Leptotrichia shahii provides interference against RNA phage. In vitro biochemical analysis shows that C2c2 is guided by a single crRNA and can be programmed to cleave ssRNA targets carrying complementary protospacers. In bacteria, C2c2 can be programmed to knock down specific mRNAs. Cleavage is mediated by catalytic residues in the two conserved HEPN domains, mutations in which generate catalytically inactive RNA-binding proteins. The RNA-focused action of C2c2 complements the CRISPR-Cas9 system, which targets DNA, the genomic blueprint for cellular identity and function. The ability to target only RNA, which helps carry out the genomic instructions, offers the ability to specifically manipulate RNA in a high-throughput manner—and manipulate gene function more broadly. These results demonstrate the capability of C2c2 as a new RNA-targeting tool.

Another Class 2 type V-B CRISPR/Cas effector “C2c1” can also be used in the present invention for editing DNA. C2c1 contains RuvC-like endonuclease domains related distantly to Cpf1 (described below). C2c1 can target and cleave both strands of target DNA site-specifically. According to PMID: 27984729, a crystal structure confirms Alicyclobacillus acidoterrestris C2c1 (AacC2c1) binds to sgRNA as a binary complex and targets DNAs as ternary complexes, thereby capturing catalytically competent conformations of AacC2c1 with both target and non-target DNA strands independently positioned within a single RuvC catalytic pocket. PMID: 27984729 confirms that C2c1-mediated cleavage results in a staggered seven-nucleotide break of target DNA, crRNA adopts a pre-ordered five-nucleotide A-form seed sequence in the binary complex, with release of an inserted tryptophan, facilitating zippering up of 20-bp guide RNA:target DNA heteroduplex on ternary complex formation, and that the PAM-interacting cleft adopts a “locked” conformation on ternary complex formation.

C2c3 is a gene editor effector of type V-C that is distantly related to C2c1, and also contains RuvC-like nuclease domains. C2c3 is also similar to the CasY.1—CasY.6 group described below.

“CRISPR Cas9” as used herein refers to Clustered Regularly Interspaced Short Palindromic Repeat (CRISPR)-associated endonuclease Cas9. In bacteria the CRISPR/Cas loci encode RNA-guided adaptive immune systems against mobile genetic elements (viruses, transposable elements and conjugative plasmids). Three types (I-III) of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA). The CRISPR-associated endonuclease, Cas9, belongs to the type II CRISPR/Cas system and has strong endonuclease activity to cut target DNA. Cas9 is guided by a mature crRNA that contains about 20 base pairs (bp) of unique target sequence (called spacer) and a trans-activated small RNA (tracrRNA) that serves as a guide for ribonuclease III-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd nucleotide from PAM). The crRNA and tracrRNA can be expressed separately or engineered into an artificial fusion small guide RNA (sgRNA) via a synthetic stem loop (AGAAAU) to mimic the natural crRNA/tracrRNA duplex. Such sgRNA, like shRNA, can be synthesized or in vitro transcribed for direct RNA transfection or expressed from U6 or H1-promoted RNA expression vector, although cleavage efficiencies of the artificial sgRNA are lower than those for systems with the crRNA and tracrRNA expressed separately.

CRISPR/Cpf1 is a DNA-editing technology analogous to the CRISPR/Cas9 system, characterized in 2015 by Feng Zhang's group from the Broad Institute and MIT. Cpfl is an RNA-guided endonuclease of a class II CRISPR/Cas system. This acquired immune mechanism is found in Prevotella and Francisella bacteria. It prevents genetic damage from viruses. Cpf1 genes are associated with the CRISPR locus, coding for an endonuclease that use a guide RNA to find and cleave viral DNA. Cpf1 is a smaller and simpler endonuclease than Cas9, overcoming some of the CRISPR/Cas9 system limitations. CRISPR/Cpf1 could have multiple applications, including treatment of genetic illnesses and degenerative conditions.

A CRISPR/TevCas9 system can also be used. In some cases, it has been shown that once CRISPR/Cas9 cuts DNA in one spot, DNA repair systems in the cells of an organism will repair the site of the cut. The TevCas9 enzyme was developed to cut DNA at two sites of the target so that it is harder for the cells' DNA repair systems to repair the cuts (PMID: 27956611). The TevCas9 nuclease is a fusion of a I-Tevi nuclease domain to Cas9.

The Cas9 nuclease can have a nucleotide sequence identical to the wild type Streptococcus pyrogenes sequence. In some embodiments, the CRISPR-associated endonuclease can be a sequence from other species, for example other Streptococcus species, such as thermophilus; Pseudomona aeruginosa, Escherichia coli, or other sequenced bacteria genomes and archaea, or other prokaryotic microorganisms. Alternatively, the wild type Streptococcus pyrogenes Cas9 sequence can be modified. The nucleic acid sequence can be codon optimized for efficient expression in mammalian cells, i.e., “humanized.” A humanized Cas9 nuclease sequence can be for example, the Cas9 nuclease sequence encoded by any of the expression vectors listed in Genbank accession numbers KM099231.1 Gl:669193757; KM099232.1 Gl:669193761; or KM099233.1 Gl:669193765. Alternatively, the Cas9 nuclease sequence can be for example, the sequence contained within a commercially available vector such as PX330 or PX260 from Addgene (Cambridge, MA). In some embodiments, the Cas9 endonuclease can have an amino acid sequence that is a variant or a fragment of any of the Cas9 endonuclease sequences of Genbank accession numbers KM099231.1 Gl:669193757; KM099232.1 Gl:669193761; or KM099233.1 Gl:669193765 or Cas9 amino acid sequence of PX330 or PX260 (Addgene, Cambridge, MA). The Cas9 nucleotide sequence can be modified to encode biologically active variants of Cas9, and these variants can have or can include, for example, an amino acid sequence that differs from a wild type Cas9 by virtue of containing one or more mutations (e.g., an addition, deletion, or substitution mutation or a combination of such mutations). One or more of the substitution mutations can be a substitution (e.g., a conservative amino acid substitution). For example, a biologically active variant of a Cas9 polypeptide can have an amino acid sequence with at least or about 50% sequence identity (e.g., at least or about 50%, 55%, 60%, 65%, 70%, 75%, 80%, 85%, 90%, 95%, 97%, 98%, or 99% sequence identity) to a wild type Cas9 polypeptide. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. The amino acid residues in the Cas9 amino acid sequence can be non-naturally occurring amino acid residues. Naturally occurring amino acid residues include those naturally encoded by the genetic code as well as non-standard amino acids (e.g., amino acids having the D-configuration instead of the L-configuration). The present peptides can also include amino acid residues that are modified versions of standard residues (e.g., pyrrolysine can be used in place of lysine and selenocysteine can be used in place of cysteine). Non-naturally occurring amino acid residues are those that have not been found in nature, but that conform to the basic formula of an amino acid and can be incorporated into a peptide. These include D-alloisoleucine (2R,3S)-2-amino-3-methylpentanoic acid and L-cyclopentyl glycine (S)-2-amino-2-cyclopentyl acetic acid. For other examples, one can consult textbooks or the worldwide web (a site is currently maintained by the California Institute of Technology and displays structures of non-natural amino acids that have been successfully incorporated into functional proteins). The Cas-9 can also be shown in TABLE 3 below.

TABLE 3

| Variant No. | | Tested* |
|---|---|---|
| | Four Alanine Substitution Mutants (compared to WT Cas9) | |
| 1 | SpCas9 N497A, R661A, Q695A, Q926A | YES |
| 2 | SpCas9 N497A, R661A, Q695A, Q926A + D1135E | YES |
| 3 | SpCas9 N497A, R661A, Q695A, Q926A + L169A | YES |
| 4 | SpCas9 N497A, R661A, Q695A, Q926A + Y450A | YES |
| 5 | SpCas9 N497A, R661A, Q695A, Q926A + M495A | Predicted |
| 6 | SpCas9 N497A, R661A, Q695A, Q926A + M694A | Predicted |
| 7 | SpCas9 N497A, R661A, Q695A, Q926A + H698A | Predicted |
| 8 | SpCas9 N497A, R661A, Q695A, Q926A + D1135E + L169A | Predicted |
| 9 | SpCas9 N497A, R661A, Q695A, Q926A + D1135E + Y450A | Predicted |
| 10 | SpCas9 N497A, R661A, Q695A, Q926A + D1135E + M495A | Predicted |
| 11 | SpCas9 N497A, R661A, Q695A, Q926A + D1135E + M694A | Predicted |
| 12 | SpCas9 N497A, R661A, Q695A, Q926A + D1135E + M698A | Predicted |
| | Three Alanine Substitution Mutants (compared to WT Cas9) | |
| 13 | SpCas9 R661A, Q695A, Q926A | No (on target only) |
| 14 | SpCas9 R661A, Q695A, Q926A + D1135E | Predicted |
| 15 | SpCas9 R661A, Q695A, Q926A + L169A | Predicted |
| 16 | SpCas9 R661A, Q695A, Q926A + Y450A | Predicted |
| 17 | SpCas9 R661A, Q695A, Q926A + M495A | Predicted |
| 18 | SpCas9 R661A, Q695A, Q926A + M694A | Predicted |
| 19 | SpCas9 R661A, Q695A, Q926A + H698A | Predicted |
| 20 | SpCas9 R661A, Q695A, Q926A + D1135E + L169A | Predicted |
| 21 | SpCas9 R661A, Q695A, Q926A + D1135E + Y450A | Predicted |
| 22 | SpCas9 R661A, Q695A, Q926A + D1135E + M495A | Predicted |
| 23 | SpCas9 R661A, Q695A, Q926A + D1135E + M694A | Predicted |

Although the RNA-guided endonuclease Cas9 has emerged as a versatile genome-editing platform, some have reported that the size of the commonly used Cas9 from Streptococcus pyogenes (SpCas9) limits its utility for basic research and therapeutic applications that use the highly versatile adeno-associated virus (AAV) delivery vehicle. Accordingly, the six smaller Cas9 orthologues have been used and reports have shown that Cas9 from Staphylococcus aureus (SaCas9) can edit the genome with efficiencies similar to those of SpCas9, while being more than 1 kilobase shorter. SaCas9 is 1053 bp, whereas SpCas9 is 1358 bp.

The Cas9 nuclease sequence, or any of the gene editor effector sequences described herein, can be a mutated sequence. For example, the Cas9 nuclease can be mutated in the conserved HNH and RuvC domains, which are involved in strand specific cleavage. For example, an aspartate-to-alanine (D10A) mutation in the RuvC catalytic domain allows the Cas9 nickase mutant (Cas9n) to nick rather than cleave DNA to yield single-stranded breaks, and the subsequent preferential repair through HDR can potentially decrease the frequency of unwanted indel mutations from off-target double-stranded breaks. In general, mutations of the gene editor effector sequence can minimize or prevent off-targeting.

The gene editor effector can be CasX or CasY or Cas Omega. CasX has a TTC PAM at the 5′ end (similar to Cpf1). The TTC PAM can have limitations in viral genomes that are GC rich, but not so much in those that are GC poor. The size of CasX (986 bp), smaller than other type V proteins, provides the potential for four gRNA plus one siRNA in a delivery plasmid. CasX can be derived from Deltaproteobacteria or Planctomycetes.

The gene editor effector can also be Archaea Cas9. The size of Archaea Cas9 is 950aa ARMAN 1 and 967aa ARMAN 4. The Archaea Cas9 can be derived from ARMAN-1 (Candidatus Micrarchaeum acidiphilum ARMAN-1) or ARMAN-4 (Candidatus Parvarchaeum acidiphilum ARMAN-4). The sequences for ARMAN 1 and ARMAN 4 are below.

In the present invention, when any of the compositions are contained within an expression vector, the CRISPR endonuclease can be encoded by the same nucleic acid or vector as the gRNA sequences. Alternatively, or in addition, the CRISPR endonuclease can be encoded in a physically separate nucleic acid from the gRNA sequences or in a separate vector.

Vectors containing nucleic acids such as those described herein are also provided. A “vector” is a replicon, such as a plasmid, phage, or cosmid, into which another DNA segment may be inserted so as to bring about the replication of the inserted segment. Generally, a vector is capable of replication when associated with the proper control elements. Suitable vector backbones include, for example, those routinely used in the art such as plasmids, viruses, artificial chromosomes, BACs, YACs, or PACs. The term “vector” includes cloning and expression vectors, as well as viral vectors and integrating vectors. An “expression vector” is a vector that includes a regulatory region. Numerous vectors and expression systems are commercially available from such corporations as Novagen (Madison, WI), Clontech (Palo Alto, CA), Stratagene (La Jolla, CA), and Invitrogen/Life Technologies (Carlsbad, CA).

The vectors provided herein also can include, for example, origins of replication, scaffold attachment regions (SARs), and/or markers. A marker gene can confer a selectable phenotype on a host cell. For example, a marker can confer biocide resistance, such as resistance to an antibiotic (e.g., kanamycin, G418, bleomycin, or hygromycin). As noted above, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag TM tag (Kodak, New Haven, CT) sequences typically are expressed as a fusion with the encoded polypeptide. Such tags can be inserted anywhere within the polypeptide, including at either the carboxyl or amino terminus.

Additional expression vectors also can include, for example, segments of chromosomal, non-chromosomal and synthetic DNA sequences. Suitable vectors include derivatives of SV40 and known bacterial plasmids, e.g., E. coli plasmids col El, pCR1, pBR322, pMal-C2, pET, pGEX, pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2p plasmid or derivatives thereof, vectors useful in eukaryotic cells, such as vectors useful in insect or mammalian cells; vectors derived from combinations of plasmids and phage DNAs, such as plasmids that have been modified to employ phage DNA or other expression control sequences.

Yeast expression systems can also be used. For example, the non-fusion pYES2 vector (Xbal, Sphl, Shol, Notl, GstXl, EcoRl, BstXl, BamH1, Sacl, Kpn1, and Hindil cloning sites; Invitrogen) or the fusion pYESHisA, B, C (Xbal, Sphl, Shol, Notl, BstXl, EcoRl, BamH1, Sacl, Kpnl, and Hindil cloning sites, N-terminal peptide purified with ProBond resin and cleaved with enterokinase; Invitrogen), to mention just two, can be employed according to the invention. A yeast two-hybrid expression system can also be prepared in accordance with the invention.

The vector can also include a regulatory region. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, nuclear localization signals, and introns.

As used herein, the term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

Vectors include, for example, viral vectors (such as adenoviruses (“Ad”), adeno-associated viruses (AAV), and vesicular stomatitis virus (VSV) and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described in PMID: 12545555. A large variety of such vectors are known in the art and are generally available.

A “recombinant viral vector” refers to a viral vector comprising one or more heterologous gene products or sequences. Since many viral vectors exhibit size-constraints associated with packaging, the heterologous gene products or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying gene products necessary for replication and/or encapsidation). Modified viral vectors in which a polynucleotide to be delivered is carried on the outside of the viral particle have also been described (PMID: 1681545).

Suitable nucleic acid delivery systems include recombinant viral vector, typically sequence from at least one of an adenovirus, adenovirus-associated virus (AAV), helper-dependent adenovirus, retrovirus, or hemagglutinating virus of Japan-liposome (HVJ) complex. In such cases, the viral vector comprises a strong eukaryotic promoter operably linked to the polynucleotide e.g., a cytomegalovirus (CMV) promoter. The recombinant viral vector can include one or more of the polynucleotides therein, preferably about one polynucleotide. In some embodiments, the viral vector used in the invention methods has a pfu (plague forming units) of from about 10⁸ to about 5×10¹⁰ pfu. In embodiments in which the polynucleotide is to be administered with a non-viral vector, use of between from about 0.1 nanograms to about 4000 micrograms will often be useful e.g., about 1 nanogram to about 100 micrograms.

Additional vectors include viral vectors, fusion proteins and chemical conjugates. Retroviral vectors include Moloney murine leukemia viruses and HIV-based viruses. One HIV-based viral vector comprises at least two vectors wherein the gag and pol genes are from an HIV genome and the env gene is from another virus. DNA viral vectors include pox vectors such as orthopox or avipox vectors, herpesvirus vectors such as a herpes simplex I virus (HSV) vector (PMID: 7830040, PMID: 8102799, PMID: 2153970,), Adenovirus Vectors (PMID: 8382374, PMID: 8387378, PMID: 7884845), and Adeno-associated Virus Vectors (PMID: 7842013).

Pox viral vectors introduce the gene into the cell's cytoplasm. Avipox virus vectors result in only a short-term expression of nucleic acid. Adenovirus vectors, adeno-associated virus vectors and herpes simplex virus (HSV) vectors may be an indication for some invention embodiments. The adenovirus vector results in a shorter-term expression (e.g., less than about a month) than adeno-associated virus, in some embodiments, may exhibit much longer expression. The particular vector chosen will depend upon the target cell and the condition being treated. The selection of appropriate promoters can readily be accomplished. An example of a suitable promoter is the 763-base-pair cytomegalovirus (CMV) promoter. Other suitable promoters which may be used for gene expression include, but are not limited to, the Rous sarcoma virus (RSV) (PMID: 8494924), the SV40 early promoter region, the herpes thymidine kinase promoter, the regulatory sequences of the metallothionein (MMT) gene, prokaryotic expression vectors such as the β-lactamase promoter, the tac promoter, promoter elements from yeast or other fungi such as the Gal 4 promoter, the ADC (alcohol dehydrogenase) promoter, PGK (phosphoglycerol kinase) promoter, alkaline phosphatase promoter; and the animal transcriptional control regions, which exhibit tissue specificity and have been utilized in transgenic animals: elastase I gene control region which is active in pancreatic acinar cells, insulin gene control region which is active in pancreatic beta cells, immunoglobulin gene control region which is active in lymphoid cells, mouse mammary tumor virus control region which is active in testicular, breast, lymphoid and mast cells, albumin gene control region which is active in liver, alpha-fetoprotein gene control region which is active in liver, alpha 1-antitrypsin gene control region which is active in the liver, beta-globin gene control region which is active in myeloid cells, myelin basic protein gene control region which is active in oligodendrocyte cells in the brain, myosin light chain-2 gene control region which is active in skeletal muscle, and gonadotropic releasing hormone gene control region which is active in the hypothalamus. Certain proteins can express using their native promoter. Other elements that can enhance expression can also be included such as an enhancer or a system that results in high levels of expression such as a tat gene and tar element. This cassette can then be inserted into a vector, e.g., a plasmid vector such as, pUC19, pUC118, pBR322, or other known plasmid vectors, that includes, for example, an E. coli origin of replication. See Sambrook, et al., Molecular Cloning: A Laboratory Manual, Cold Spring Harbor Laboratory press, (1989). The plasmid vector may also include a selectable marker such as the 8-lactamase gene for ampicillin resistance, provided that the marker polypeptide does not adversely affect the metabolism of the organism being treated. The cassette can also be bound to a nucleic acid binding moiety in a synthetic delivery system, such as the system disclosed in WO 95/22618.

If desired, the polynucleotides of the invention can also be used with a microdelivery vehicle such as cationic liposomes and adenoviral vectors. For a review of the procedures for liposome preparation, targeting and delivery of contents, see PMID: 2856201. See also, Feigner and Holm, Bethesda Res. Lab. Focus, 11(2):21 (1989) and Maurer, R.A., Bethesda Res. Lab. Focus, 11(2):25 (1989).

Replication-defective recombinant adenoviral vectors can be produced in accordance with known techniques. See PMID: 1557362, PMID: 1644927, and PMID: 1370653.

Another delivery method is to use single stranded DNA producing vectors which can produce the expressed products intracellularly. See for example, PMID: 12545555), which is incorporated herein, by reference, in its entirety. Alternatively, RNA and/or protein therapeutic delivery can also be used.

As described above, the compositions of the present invention can be prepared in a variety of ways known to one of ordinary skill in the art. Regardless of their original source or the manner in which they are obtained, the compositions of the invention can be formulated in accordance with their use. For example, the nucleic acids and vectors described above can be formulated within compositions for application to cells in tissue culture or for administration to a patient or subject. Any of the pharmaceutical compositions of the invention can be formulated for use in the preparation of a medicament, and particular uses are indicated below in the context of treatment. When employed as pharmaceuticals, any of the nucleic acids and vectors can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intra-arterial, subcutaneous, intraperitoneal or intramuscular injection or infusion; or intracranial, e.g., intrathecal or intraventricular administration. Parenteral administration can be in the form of a single bolus dose, or maybe, for example, by a continuous perfusion pump. Pharmaceutical compositions and formulations for topical administration may include transdermal patches, ointments, lotions, creams, gels, drops, suppositories, sprays, liquids, powders, and the like. Conventional pharmaceutical carriers, aqueous, powder or oily bases, thickeners and the like may be necessary or desirable.

This invention also includes pharmaceutical compositions which contain, as the active ingredient, nucleic acids and vectors described herein in combination with one or more pharmaceutically acceptable carriers. We use the terms “pharmaceutically acceptable” (or “pharmacologically acceptable”) to refer to molecular entities and compositions that do not produce an adverse, allergic or other untoward reaction when administered to an animal or a human, as appropriate. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance. In making the compositions of the invention, the active ingredient is typically mixed with an excipient, diluted by an excipient or enclosed within such a carrier in the form of, for example, a capsule, tablet, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), lotions, creams, ointments, gels, soft and hard gelatin capsules, suppositories, sterile injectable solutions, and sterile packaged powders. As is known in the art, the type of diluent can vary depending upon the intended route of administration. The resulting compositions can include additional agents, such as preservatives. In some embodiments, the carrier can be, or can include a lipid-based or polymer-based colloid. In some embodiments, the carrier material can be a colloid formulated as a liposome, a hydrogel, a microparticle, a nanoparticle, or a block copolymer micelle. As noted, the carrier material can form a capsule, and that material may be a polymer-based colloid.

The nucleic acid sequences of the invention can be delivered to an appropriate cell of a subject. This can be achieved by, for example, the use of a polymeric, biodegradable microparticle or microcapsule delivery vehicle, sized to optimize phagocytosis by phagocytic cells such as macrophages. For example, PLGA (poly-lacto-co-glycolide) microparticles approximately 1-10 μm in diameter can be used. The polynucleotide is encapsulated in these microparticles, which are taken up by macrophages and gradually biodegraded within the cell, thereby releasing the polynucleotide. Once released, the DNA is expressed within the cell. A second type of microparticle is intended not to be taken up directly by cells, but rather to serve primarily as a slow-release reservoir of nucleic acid that is taken up by cells only upon release from the micro-particle through biodegradation. These polymeric particles should therefore be large enough to preclude phagocytosis (i.e., larger than 5 μm and preferably larger than 20 μm). Another way to achieve uptake of the nucleic acid is using liposomes, prepared by standard methods. The nucleic acids can be incorporated alone into these delivery vehicles or co-incorporated with tissue-specific antibodies, for example antibodies that target cell types that are commonly latently infected reservoirs of viral infection, for example, brain macrophages, microglia, astrocytes, and gut-associated lymphoid cells. Alternatively, one can prepare a molecular complex composed of a plasmid or other vector attached to poly-L-lysine by electrostatic or covalent forces. Poly-L-lysine binds to a ligand that can bind to a receptor on target cells. Delivery of “naked DNA” (i.e., without a delivery vehicle) to an intramuscular, intradermal, or subcutaneous site, is another means to achieve in vivo expression. In the relevant polynucleotides (e.g., expression vectors) the nucleic acid sequence encoding an isolated nucleic acid sequence comprising a sequence encoding a CRISPR-associated endonuclease and a guide RNA is operatively linked to a promoter or enhancer-promoter combination. Promoters and enhancers are described above.

In some embodiments, the compositions of the invention can be formulated as a nanoparticle, for example, nanoparticles comprised of a core of high molecular weight linear polyethylenimine (LPEI) complexed with DNA and surrounded by a shell of polyethyleneglycol-modified (PEGylated) low molecular weight LPEI.

The nucleic acids and vectors may also be applied to a surface of a device (e.g., a catheter) or contained within a pump, patch, or other drug delivery device. The nucleic acids and vectors of the invention can be administered alone, or in a mixture, in the presence of a pharmaceutically acceptable excipient or carrier (e.g., physiological saline). The excipient or carrier is selected on the basis of the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

The methods of the invention can be expressed in terms of the preparation of a medicament. Accordingly, the invention encompasses the use of the agents and compositions described herein in the preparation of a medicament. The compounds described herein are useful in therapeutic compositions and regimens or for the manufacture of a medicament for use in treatment of diseases or conditions as described herein.

Any composition described herein can be administered to any part of the host's body for subsequent delivery to a target cell. A composition can be delivered to, without limitation, the brain, the cerebrospinal fluid, joints, nasal mucosa, blood, lungs, intestines, muscle tissues, skin, or the peritoneal cavity of a mammal. In terms of routes of delivery, a composition can be administered by intravenous, intracranial, intraperitoneal, intramuscular, subcutaneous, intramuscular, intrarectal, intravaginal, intrathecal, intratracheal, intradermal, or transdermal injection, by oral or nasal administration, or by gradual perfusion over time. In a further example, an aerosol preparation of a composition can be given to a host by inhalation.

The dosage required will depend on the route of administration, the nature of the formulation, the nature of the animal's illness, the animal's size, weight, surface area, age, and sex, other drugs being administered, and the judgment of the attending clinicians. Wide variations in the needed dosage are to be expected in view of the variety of cellular targets and the differing efficiencies of various routes of administration. Variations in these dosage levels can be adjusted using standard empirical routines for optimization, as is well understood in the art. Administrations can be single or multiple (e.g., 2- or 3-, 4-, 6-, 8-, 10-, 20-, 50-, 100-, 150-, or more fold). Encapsulation of the compounds in a suitable delivery vehicle (e.g., polymeric microparticles or implantable devices) may increase the efficiency of delivery. Dosage can be given to provide total viral load elimination. Dosage can also be given to reduce viral load within the animal to allow for the immune destruction of the remainder of the viral load.

The duration of treatment with any composition provided herein can be any length of time from as short as one day to as long as the life span of the host (e.g., many years). For example, a compound can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compounds can be administered once (or twice, three times, etc.) daily, weekly, monthly, or yearly.

An effective amount of any composition provided herein can be administered to an individual in need of treatment. The term “effective” as used herein refers to any amount that induces a desired response while not inducing significant toxicity in the patient. Such an amount can be determined by assessing a patient's response after administration of a known amount of a particular composition. In addition, the level of toxicity, if any, can be determined by assessing an individual's clinical symptoms before and after administering a known amount of a particular composition. It is noted that the effective amount of a particular composition administered to a patient can be adjusted according to a desired outcome as well as the individual's response and level of toxicity. Significant toxicity can vary for each particular individual and depends on multiple factors including, without limitation, the individual's disease state, age, and tolerance to side effects.

Any method known to those in the art can be used to determine if a particular response is induced. Clinical methods that can assess the degree of a particular disease state can be used to determine if a response is induced. The particular methods used to evaluate a response will depend upon the nature of the individual's disorder, the individual's age, and sex, other drugs being administered, and the judgment of the attending clinician. The viral load in the individual can be monitored, for example, with a blood test that measures viral RNA per milliliter of blood. Examples of such tests include quantitative branched DNA (bDNA), reverse transcriptase-polymerase chain reaction (RT-PCR), and qualitative transcription-mediated amplification.

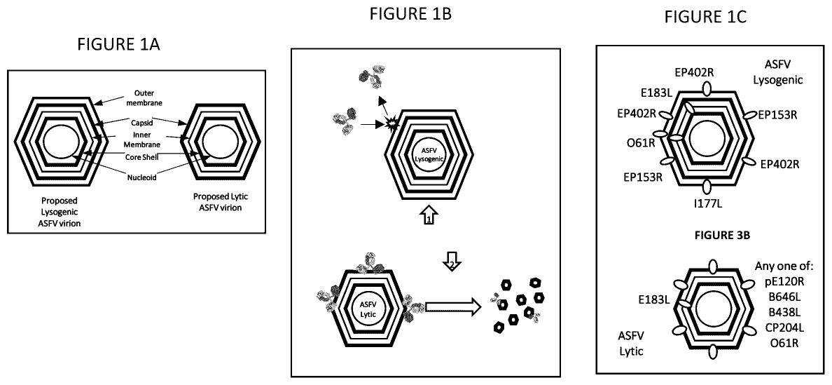

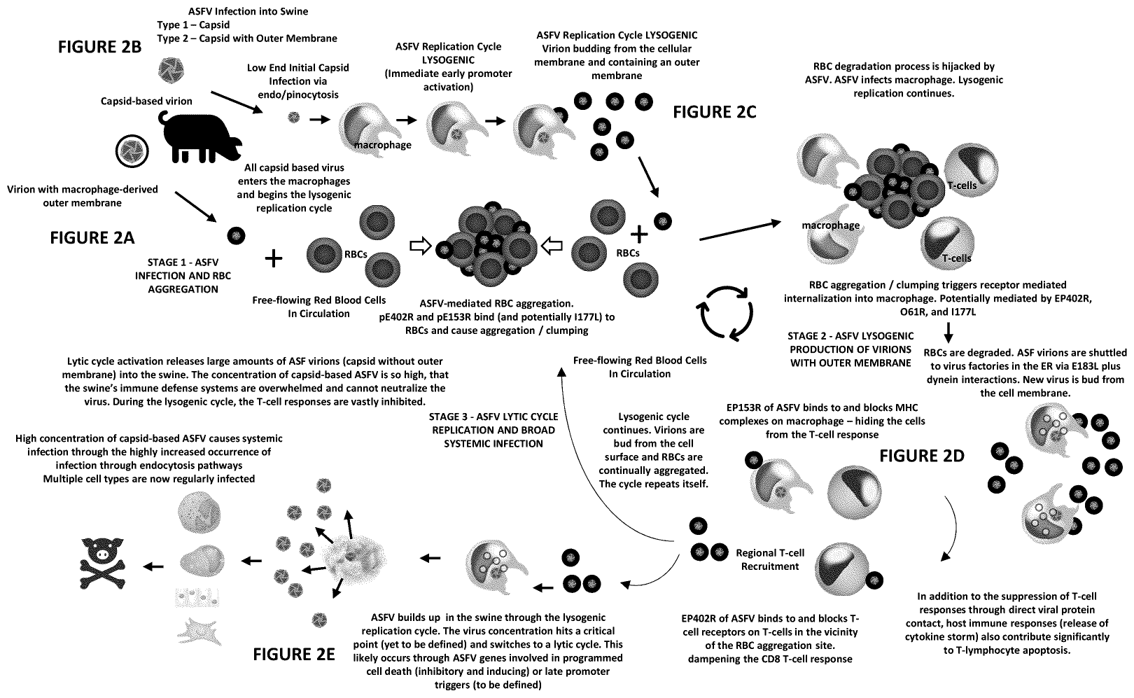

The present invention also provides for specific methods of treating ASFV. It is hypothesized that ASFV is both lytic and lysogenic (FIGS. 1A-1C and FIGS. 2A-2E). In the early stages of the virus, it is likely locked into a lysogenic replication cycle, where it buds from the monocyte/macrophage cell membrane resulting in an ASFV particle that is surrounded by an outer membrane lipid bilayer containing both viral and host cell proteins (FIG. 2D). As the virus spreads through the body of the swine, it is hypothesized that something shifts the lysogenic cycle to a lytic cycle (mechanism undefined) (FIG. 2E). During the lytic cycle, the infected cells burst, sending ASFV (without an outer membrane, capsid only) into the infected swine's body, shown in FIG. 2E. It has been shown that both types of ASFV virion are infectious (FIGS. 2A and 2B).

Antibody and antigen-based vaccines have not worked, and it is likely because the strategies for their development have not taken into account both types of replication cycle—lysogenic and lytic. For example, antibodies targeting the capsid protein (antibodies that are either directly injected as a therapeutic, or antibodies that are stimulated in vivo from an immune response to a viral peptide) may not neutralize the ASFV virion because the capsid is protected by an outer membrane (i.e., it is inaccessible). As the viral replication cycle shifts to a lytic cycle, the antibodies may indeed interact with their respective capsid epitopes, but at this stage, the in vivo viral titre is likely too high to have effective and lasting neutralizing responses. This, combined with rapid viral expansion (correlated with a 24- to 72-hour 100% mortality rate associated with the virus) contributes to the body being overwhelmed by virus. Antigen stimulation as a preventative has not worked in the past as the antigens are almost always capsid-based. Therefore, the B-cell response producing the IgG does not recognize early ASFV that is surrounded by an outer membrane (shown in FIG. 1B and FIG. 2E).

Therefore, the present invention provides for a method of treating a viral infection in an individual with a virus that is both lysogenic and lytic, by administering a viral antigen (to stimulate a B-cell response) and/or antibody therapeutic that targets protein on an outer membrane of a lysogenic phase of the virus, administering a viral antigen (to stimulate a B-cell response) and/or antibody therapeutic that targets protein on a capsid of a lytic phase of the virus, and treating the viral infection (FIG. 4).

To overcome these challenges a new two-fold strategy is necessary.

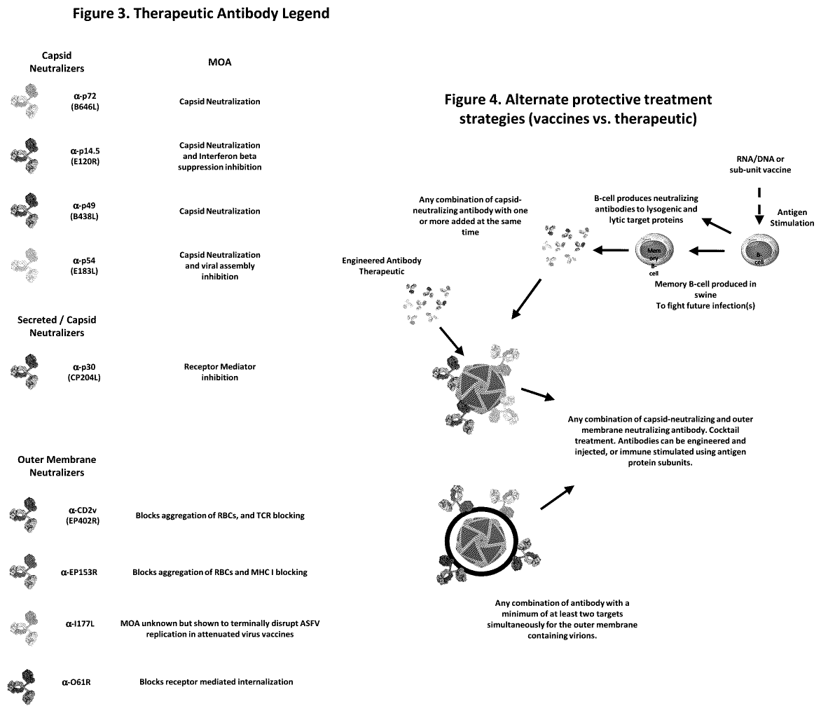

1. The swine needs to be stimulated with a viral antigen derived from a viral protein that exists on the outer membrane of the ASFV to neutralize virions that bud (lysogenic) in early infection. These viral antigens include EP402R (CD2v), EP153R, p21 (063L), and I177L (but not limited to these outer membrane proteins, per strategy) which are viral outer membrane proteins. Further, E183L (p54) is an integral viral inner membrane protein that plays a critical role in virions trafficking to viral factories in the ER during the early lysogenic stages of viral replication, as mentioned in detail above (FIG. 3 and FIG. 4).

2. The swine also needs to be treated with a viral antigen derived from a viral protein that exists on the capsid to neutralize virions that do not have an outer membrane (lytic) late infection. These viral antigens can include at least one type of capsid protein such as, p30 (CP204L), p72 (B646L), p49 (B438L), and other capsid proteins that are accessible to antibody neutralization and elicit a strong immune response (FIG. 3 and FIG. 4).

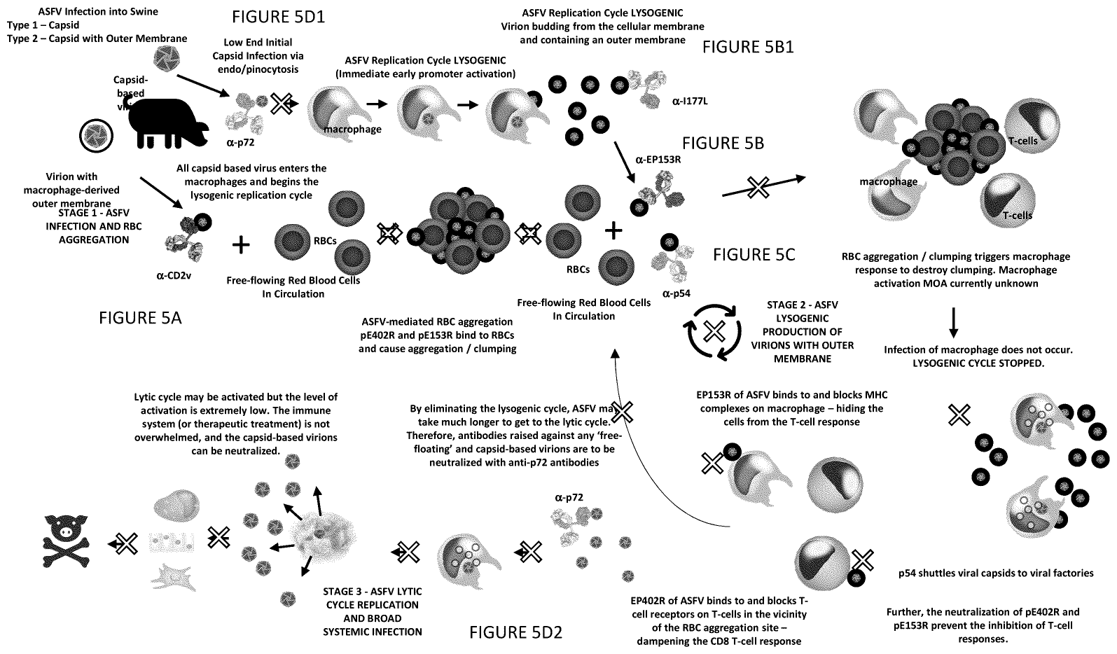

Recently, two viral outer membrane proteins were identified that is responsible for the extracellular viral docking with erythrocytes (a likely mechanism to rapidly distribute the virus through the circulating blood), T-cell suppression and MCH Class I blocking. These proteins are called EP402R (CD2v) and EP153R. EP402R has also been shown to be responsible for immunosuppressive activity by inhibiting lymphocyte proliferation. EP153R has been shown to be responsible for blocking the MCH Class I complexes on infected macrophages. Therefore, by targeting the EP402R (CD2v) and EP153R protein for vaccine or therapeutic purposes, the extracellular virus will be greatly inhibited to spread throughout the organism as well as prevent lymphocyte inhibition (shown in FIGS. 5A through 5D2).

Other key proteins for viral structure that compose the capsid include p14.5 (E120R), p72 (B646L), p30 (CP204L), and p49 (B438L). These three proteins can be targeted for vaccine and/or therapeutic approaches, in order to neutralize the extracellular virions that lack an outer membrane (as result of the lytic cycle) (shown in FIGS. 5A through 5D2)).

The swine can therefore be treated with either whole proteins, or a peptide (surface exposed), or a mixture of peptides derived from: 1) the proteins involved in the early stage lysogenic cycle of ASFV replication such as i) the outer membrane proteins EP402R (CD2v) and EP153R and/or ii) the integral viral inner membrane protein E183L (p54), and in combination with 2) the proteins involved in the lytic cycle of ASFV replication such as i)p14.5 (E120R), p72 (B646L), p49 (B438L) and/or ii) the inner and outer membrane protein p12 (016R. The treatment produces a B-cell response (immediate and memory) in the swine as a prophylactic measure against ASFV lysogenic and lytic replication cycles. Peptide segments of any of these proteins (and not whole protein) can be used to create an immune stimulating response. This strategy is shown in FIGS. 5A through 5D2)

The present invention also provides for a composition for treating a viral infection in an individual with a virus that is both lysogenic and lytic including a viral antigen (that stimulates a B-cell response) and/or antibody therapeutic that targets protein on an outer membrane of a lysogenic phase of the virus and a viral antigen that targets protein on a capsid of a lytic phase of the virus (FIG. 4).

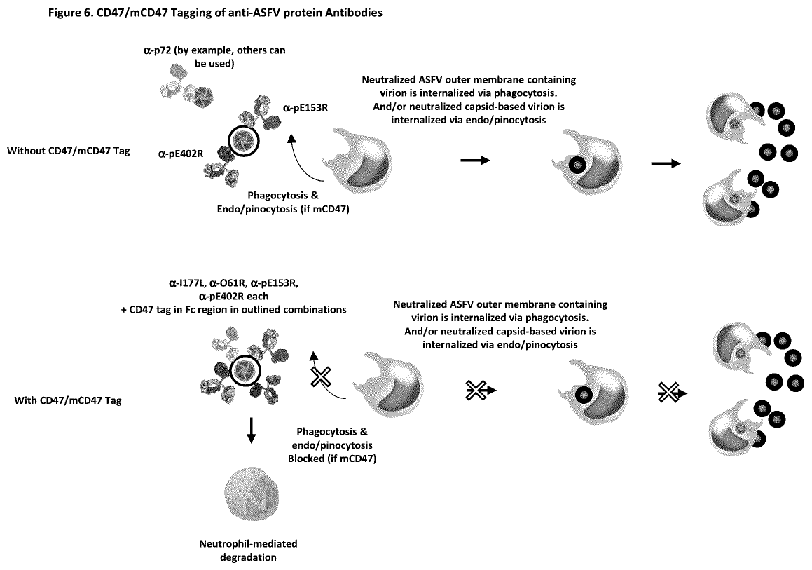

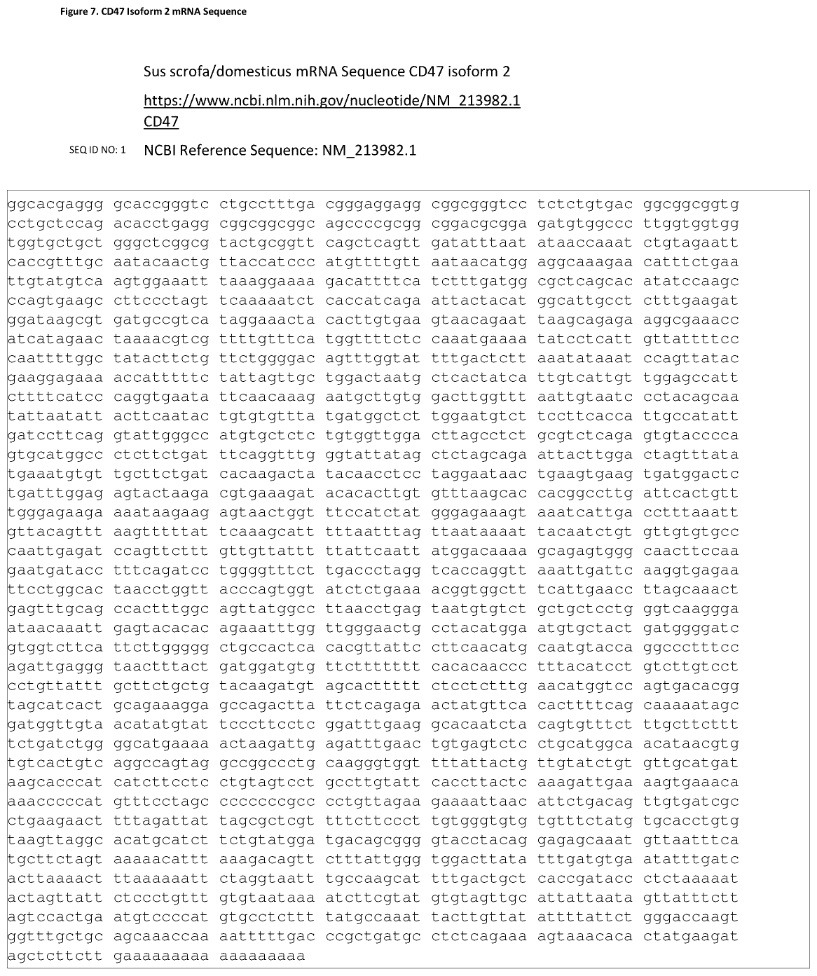

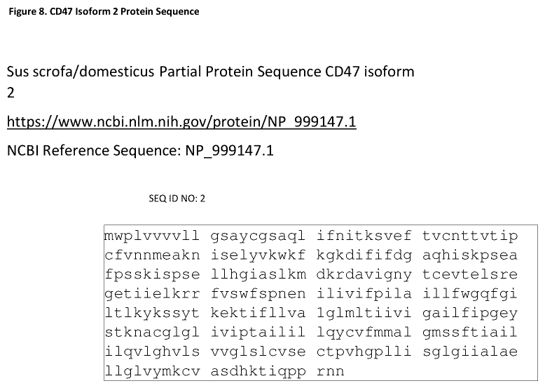

The most optimal antibodies and epitope sequences can be found for each of the proteins that define the lysogenic and lytic stages of the virus using screening or Al based approaches. Once the antibodies are defined, they can be manufactured and injected into healthy individuals (i.e., swine) to protect them from ASFV infection. Alternatively, once the antibody epitopes are defined, they can be used to engineer new antibodies, such as 1) a bispecific antibody that recognizes two viral epitopes and therefore neutralizes multiple points of the virus. The two epitopes (if bispecific they can also be used to target a protein of the lysogenic cycle and a protein of the lytic cycle simultaneously, and/or 2) the addition of CD47/mCD47 to the Fc region of the antibody (FIG. 6).

The treatment can include an antigen stimulation approach using at least one of:
1. Two separate injections, one each with peptides of CD2v (EP402R), EP153R, p54 (E183L), I177L, p12 (061R) followed by whole protein pE120R, p72 (B646L), p30 (CP204L), p49 (B438L) or p12 (061R), or any other capsid protein that may elicit and strong immune response.
2. Two separate injections of a peptide segment derived from each of CD2v (EP402R), EP153R, p54 (E183L), I177L, p12 (061R) or pE120R, p72 (B646L), p30 (CP204L), p49 (B438L), p12 (061R). The peptide(s) can be derived from an epitope that is exposed on the outer surface of either the outer membrane or the capsid.
3. Two separate injections of a pool of peptide(s) segments derived from each of CD2v (EP402R), EP153R, p54 (E183L), I177L, p12 (061R) or pE120R, p72 (B646L), p30 (CP204L), p49 (B438L), p12 (061R). The peptide pool will be derived from epitopes that are exposed on the outer surface of either the outer membrane or the capsid.
4. One injection containing one each with peptides of CD2v (EP402R), EP153R, p54 (E183L), I177L, p12 (061) followed by whole protein pE120R, p72 (B646L), p30 (CP204L), p49 (B438L), p12 (061R).
5. One injection containing a peptide segment derived from each of CD2v (EP402R), EP153R, p54 (E183L), I177R, p12 (061R) and pE120R, p72 (B646L), p30 (CP204L), p49 (B438L), p12 (061R). Peptides can be derived from an epitope that is exposed on the outer surface of either the outer membrane or the capsid.
6. One injection containing a pool of peptide segments derived from each of CD2v (EP402R), EP153R, p54 (E183L), I177L, p12 (061R) or p14.5 (E120R), p72 (B646L), p30 (CP204L), p49 (B438L), p12 (061R). The peptide pool can be derived from epitopes that are exposed on the outer surface of either the outer membrane or the capsid.

Each of these strategies are not limited to CD2v (EP402R), EP153R, p54 (E183L), I177L pE120R, p72 (B646L), p30 (CP204L), p49 (B438L), or p12 (061R) additional outer membrane and capsid proteins can be exploited for the same purpose/outcome.

CD2v (EP402R), EP153R, p54 (E183L), I177L and/or pE120R, p72 (B646L), p30 (CP204L), p49 (B438L), p12 (061R) whole proteins or any combination of peptide(s) thereof, can be used as antigens to discover antibodies using any type of antibody discovery platform. Some of these platforms include gene editing-driven antibody over-expression systems in B-cells, phage libraries, yeast expression systems, nano well GFP-labeling systems, to name a few. Once the antibodies are discovered, they can be tested for affinity, avidity, specificity, selectivity, stability, precision, robustness, and the best candidates (derived from a platform screen) can be used as a therapeutic treatment to neutralize viral CD2v (EP402R), EP153R, p54 (E183L), I177L and/or pE120R, p72 (B646L), p30 (CP204L), p49 (B438L), p12 (061R) (or other outer membrane and/or capsid proteins) after the swine have been infected.

The therapeutic treatment can include at least one of: two separate injections, one each of an antibody (or several neutralizing antibodies) raised against CD2v (EP402R), EP153R, p54 (E183L), I177L and/or pE120R, p72 (B646L), p30 (CP204L), p49 (B438L), p12 (061R); or one injection containing a pool of antibodies raised against CD2v (EP402R), EP153R, p54 (E183L), I177L and/or pE120R, p72 (B646L), p30 (CP204L), p49 (B438L), p12 (061R). This strategy can also be used in treating humans if the virus jumps species.

Therefore, the present invention provides for a method of finding antibodies for treating a viral infection in an individual with a virus that is both lysogenic and lytic, by using whole proteins or peptides of target protein on an outer membrane of a lysogenic phase of the virus and target protein on a capsid of a lytic phase of the virus as antigens to discover antibodies with an antibody discovery platform, testing discovered antibodies for affinity, avidity, specificity, selectivity, stability, precision, and robustness, and selecting a best candidate antibody as a therapeutic treatment for the viral infection. The present invention also provides for the antibodies found by this method.

The present invention also provides for a vaccine for preventing viral infection, including whole and/or partial domains of proteins of both a lysogenic and lytic phase of a virus. The domains can include at least 50%, 55%, 60%, 65%, 70%, 75%, 80%, 85%, 90%, 95%, 97%, 98%, or 99% sequence identity similarity. The proteins can be expressed using an mRNA deliverable to stimulate B-cells in the individual to produce the proteins and corresponding neutralizing antibodies.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

### REFERENCES

1. Barasona et al. First Oral Vaccination of Eurasian Wild Boar against African Swine Fever Virus Genotype II. Front. Vet. Sci. 2019, 6, 137. PMID: 31106218
2. Gallardo et al. African swine fever virus (ASFV) protection mediated by NH/P68 and NH/P68 recombinant live-attenuated viruses. Vaccine 2018, 36, 2694-2704. PMID: 29609966
3. O'Donnell et al Simultaneous deletion of the 9GL and UK genes from the African swine fever virus Georgia 2007 isolate offers increased safety and protection against homologous challenge. J. Virol. 2017, 91, e01760-16. PMID: 27795430
4. Monteagudo et al. BA71ACD2: A new recombinant live attenuated African swine fever virus with cross-protective capabilities. J. Virol. 2017, 91, e01058-17. PMID: 28814514
5. Lopez et al. Live Attenuated African Swine Fever Viruses as Ideal Tools to Dissect the Mechanisms Involved in Cross-Protection. Viruses 2020, 12, 1474. PMID: 33371460
6. Chen et al. A seven-gene-deleted African swine fever virus is safe and effective as a live attenuated vaccine in pigs. Sci. China Life Sci. 2020, 63, 623-634. PMID: 32124180
7. Borca et al. Development of a highly effective African swine fever virus vaccine by deletion of the I177L gene results in sterile immunity against the current epidemic Eurasia strain. J. Virol. 2020, 94, e02017-19. PMID: 31969432
8. Teklue et al, Generation and evaluation of an African swine fever virus mutant with deletion of the CD2v and UK genes. Vaccines 2020, 8, 763. PMID: 33327488
9. Alejo et al. A proteomic atlas of the African swine fever virus particle. J. Virol. 2018, 92, e01293-18. PMID: 30185597
10. Liu et al. Cryo-EM structure of the African swine fever virus. Cell Host Microbe 2019, 26, 836-843. PMID: 31787524
11. HObner et al. Efficient inhibition of African swine fever virus replication by CRISPR/Cas9 targeting of the viral p30 gene (CP204L). Sci Rep. 2018 Jan. 23; 8(1):1449. PMID: 29362418
12. Borca et al. CRISPR/Cas Gene Editing of a Large DNA Virus: African Swine Fever Virus. Bio Protoc. 2018 Aug. 20; 8(16):e2978. PMID: 34395778
13. Wo2niakowski et al. Attempts at the Development of a Recombinant African Swine Fever Virus Strain with Abrogated EP402R, 9GL, and A238L Gene Structure using the CRISPR/Cas9 System. J Vet Res. 2020 Jun. 3; 64(2):197-205. PMID: 32587905
14. Petrovan et al. Role of African swine fever virus (ASFV) proteins EP153R and EP402R in reducing viral persistence in blood and virulence in pigs infected with BenindeltaDP148R. J Virol. 2021 Oct 13:JV10134021. PMID: 34643433
15. Ruiz-Gonzalvo et al. Functional and immunological properties of the baculovirus-expressed hemagglutinin of African swine fever virus. Virology. 1996 Apr. 1; 218(1):285-9. PMID: 8615037
16. Neilan et al. Neutralizing antibodies to African swine fever virus proteins p30, p54, and p72 are not sufficient for antibody-mediated protection. Virology. 2004 Feb. 20; 319(2):337-42. PMID: 14980493
17. Lopera-Madrid et al. Safety and immunogenicity of mammalian cell derived and Modified Vaccinia Ankara vectored African swine fever subunit antigens in swine. Vet Immunol Immunopathol. 2017 Mar;185:20-33. PMID: 28241999
18. Borca et al. ASFV-G-41177L as an Effective Oral Nasal Vaccine against the Eurasia Strain of Africa Swine Fever. Viruses. 2021 Apr. 27; 13(5):765. PMID: 33925435
19. Netherton et al. Identification and Immunogenicity of African Swine Fever Virus Antigens. Front Immunol. 2019 Jun. 19; 10:1318. PMID: 31275307
20. Wang et al. Architecture of African swine fever virus and implications for viral assembly. Science. 2019 Nov 1;366(6465):640-644. PMID: 31624094
21. Yang et al. Identification of a new cell-penetrating peptide derived from the african swine fever virus CD2v protein. Drug Deliv. 2021 December; 28(1):957-962. PMID: 34006158
22. Zhang et al. Antigenicity and immunogenicity of recombinant proteins comprising African swine fever virus proteins p30 and p54 fused to a cell-penetrating peptide. Int Immunopharmacol. 2021 Oct. 26; 101(Pt A):108251. doi: 10.1016/j.intimp.2021.108251. Online ahead of print. PMID: 34715492
23. Tesfagaber et al. Characterization of Anti-p54 Monoclonal Antibodies and Their Potential Use for African Swine Fever Virus Diagnosis. Pathogens. 2021 Feb. 7; 10(2)178. PMID: 33562314
24. Hernaez et al. The African swine fever virus dynein-binding protein p54 induces infected cell apoptosis. FEBS Lett. 2004 Jul. 2; 569(1-3):224-8. PMID: 15225638
25. Chen et al. Porcine Immunoglobulin Fc Fused P30/P54 Protein of African Swine Fever Virus Displaying on Surface of S. cerevisiae Elicit Strong Antibody Production in Swine. Virol Sin. 2021 Apr;36(2):207-219. PMID: 32915442
26. Russ et al. Blocking “don't eat me” signal of CD47-SIRPα in hematological malignancies, an in-depth review. Blood Rev. 2018 November; 32(6):480-489. PMID: 29709247
27. Csanyi et al. CD47 and Nox1 Mediate Dynamic Fluid-Phase Macropinocytosis of Native LDL. Antioxid Redox Signal. 2017 Jun. 1; 26(16):886-901. PMID: 27958762
28. Sanchez et al. Mechanisms of Entry and Endosomal Pathway of African Swine Fever Virus. Vaccines (Basel). 2017 Nov. 8; 5(4):42. PMID: 29117102
29. Sanchez et al. African swine fever virus uses macropinocytosis to enter host cells. PLoS Pathog. 2012; 8(6):e1002754. PMID: 22719252
30. Angulo et al. Virus-host interactions in African swine fever: the attachment to cellular receptors. Arch Virol Suppl. 1993; 7:169-83. Review. PMID: 8219802
31. Galindo et al. Protein cell receptors mediate the saturable interaction of African swine fever virus attachment protein p12 with the surface of permissive cells. Virus Res. 1997 June;49(2):193-204. PMID: 9213394
32. Gallardo et al. African swine fever virus (ASFV) protection mediated by NH/P68 and NH/P68 recombinant live-attenuated viruses. Vaccine. 2018 May 3;36(19):2694-2704. PMID: 29609966
33. Hurtado et al. The African swine fever virus lectin EP153R modulates the surface membrane expression of MHC class I antigens. Arch Virol. 2011 February;156(2):219-34. PMID: 21069396
34. Jia et al. Roles of African Swine Fever Virus Structural Proteins in Viral Infection. J Vet Res. 2017 Dec 6;61(2):135-143. PMID: 29978065
35. Brun et al. African swine fever virus gene Al 79L, a viral homologue of bcl-2, protects cells from programmed cell death. Virology. 1996 Nov. 1; 225(1):227-30. PMID: 8918551
36. Afonso et al. An African swine fever virus Bc1-2 homolog, 5-HL, suppresses apoptotic cell death. J Virol. 1996 July;70(7):4858-63. PMID: 8676523
37. Oura et al. African swine fever: a disease characterized by apoptosis. J Gen Virol. 1998 June;79 Pt 6):1427-38. PMID: 9634085
38. Zsak et al. Regulation of apoptosis in African swine fever virus-infected macrophages. Scientific World Journal. 2002 May 3; 2:1186-95. PMID: 12805900
39. Nogal et al. African swine fever virus IAP homologue inhibits caspase activation and promotes cell survival in mammalian cells. J Virol. 2001 March;75(6):2535-43. PMID: 11222676
40. GOrnez del Moral et al. African swine fever virus infection induces tumor necrosis factor alpha production: implications in pathogenesis. J Virol. 1999 March;73(3):2173-80. PMID: 9971800
41. Galindo et al. Protein cell receptors mediate the saturable interaction of African swine fever virus attachment protein p12 with the surface of permissive cells. Virus Res. 1997 Jun;49(2)193-204. PMID: 9213394
42. Wardley et al. The growth of virulent African swine fever virus in pig monocytes and macrophages. J Gen Virol. 1978 January;38(1):183-6. PMID: 340610
43. Goatley et al. Processing and localization of the African swine fever virus CD2v transmembrane protein. Virol. 2011 April; 85(7):3294-305.PMID: 21248037
44. Ruiz-Gonzalvo et al. Characterization of a soluble hemagglutinin induced in African swine fever virus-infected cells. 1993 Oct;196(2):769-77. PMID: 8372447
45. Knudsen et al. Cytopathogenic effect of African swine fever virus for pig monocytes: characterization and use in microassay. Vet Microbiol. 1987 May; 14(1):15-24. PMID: 3307125
46. Valdeira et al. Morphological study on the entry of African swine fever virus into cells. Biol Cell. 1985;55(1-2):35-40. PMID: 2937495
47. Valdeira et al. Entry of African swine fever virus into Vero cells and uncoating. Vet Microbiol. 1998 Feb 28;60(2-4):131-40. PMID: 9646445
48. Angulo et al. Virus-host interactions in African swine fever: the attachment to cellular receptors. Arch Virol Suppl. 1993; 7:169-83. PMID: 8219802 Review.
49. Galindo et al. African swine fever virus infects macrophages, the natural host cells, via clathrin- and cholesterol-dependent endocytosis. Virus Res. 2015 Mar. 16; 200:45-55. PMID: 25662020
50. Hernaez et al. African Swine Fever Virus Undergoes Outer Envelope Disruption, Capsid Disassembly and Inner Envelope Fusion before Core Release from Multivesicular Endosomes. PLoS Pathog. 2016 Apr. 25; 12(4):e1005595. PMID: 27110717
51. Sanchez et al. Mechanisms of Entry and Endosomal Pathway of African Swine Fever Virus. Vaccines (Basel). 2017 Nov. 8; 5(4):42. PMID: 29117102
52. Matamoros et al. African Swine Fever Virus Protein pE199L Mediates Virus Entry by Enabling Membrane Fusion and Core Penetration. G.mBio. 2020 Aug. 11; 11(4):e00789-20. PMID: 32788374
53. Sanchez et al. African swine fever virus uses macropinocytosis to enter host cells. PLoS Pathog. 2012; 8(6):e1002754. PMID: 22719252
54. Rodriguez et al. African swine fever virus structural protein p54 is essential for the recruitment of envelope precursors to assembly sites. J Virol. 2004 April; 78(8):4299-1313. PMID: 15047843
55. Liu et al. Cryo-EM Structure of the African Swine Fever Virus. Cell Host Microbe. 2019 Dec. 11; 26(6):836-843.e3. PMID: 31787524
56. Hurtado et al. The African swine fever virus lectin EP153R modulates the surface membrane expression of MHC class I antigens. Arch Virol. 2011 February;156(2):219-34. PMID: 21069396
57. Cackett et al. The African Swine Fever Virus Transcriptome. Virol. 2020 Apr 16;94(9):e00119-20. PMID: 32075923
58. Petrovan et al. Role of African Swine Fever Virus Proteins EP153R and EP402R in Reducing Viral Persistence in Blood and Virulence in Pigs Infected with BeninADP148R. J Virol. 2022 Jan. 12; 96(1):e0134021. PMID: 34643433
59. Cole et al. Membrane fusion mediated by herpesvirus glycoproteins: the paradigm of varicella-zoster virus. Rev Med Virol. 2003 Jul-Aug;13(4):207-22. PMID: 12820183
60. Langosch et al. The role of transmembrane domains in membrane fusion. Cell Mol Life Sci. 2007 Apr;64(7-8):850-64. PMID: 17429580
61. Chen et al. Expression of ssDNA in mammalian cells. Biotechniques. 2003 January;34(1):167-71. PMID: 12545555.
62. Latour et al. Bidirectional negative regulation of human T and dendritic cells by CD47 and its cognate receptor signal-regulator protein-alpha: down-regulation of IL-12 responsiveness and inhibition of dendritic cell activation. J Immunol. 2001 Sep. 1; 167(5):2547-54. PMID: 11509594
63. Rees et al. Base editing: precision chemistry on the genome and transcriptome of living cells. Review Nat Rev Genet. 2018 December; 19(12):770-788. PMID: 30323312
64. Zheng et al. Highly efficient base editing in bacteria using a Cas9-cytidine deaminase fusion. Commun Biol. 2018 Apr. 19; 1:32. PMID: 30271918
65. Yang et al. PAM-Dependent Target DNA Recognition and Cleavage by C2c1 CRISPR-Cas Endonuclease. Cell. 2016 Dec. 15; 167(7):1814-1828.e12. PMID: 27984729
66. Wolfs et al. Biasing genome-editing events toward precise length deletions with an RNA-guided TevCas9 dual nuclease, Proc Natl Acad Sci USA. 2016 Dec. 27; 113(52):14988-14993. PMID: 27956611
67. Curiel et al. Adenovirus enhancement of transferrin-polylysine-mediated gene delivery. Proc Natl Acad Sci USA. 1991 Oct. 1; 88(19):8850-4. PMID: 1681545
68. Geller et al. An HSV-1 vector expressing tyrosine hydroxylase causes production and release of L-dopa from cultured rat striatal cells. J Neurochem. 1995 February;64(2):487-96. PMID: 7830040
69. Geller et al. Long-term increases in neurotransmitter release from neuronal cells expressing a constitutively active adenylate cyclase from a herpes simplex virus type 1 vector. Proc Natl Acad Sci USA. 1993 Aug. 15; 90(16):7603-7. PMID: 8102799
70. Geller et al. Infection of cultured central nervous system neurons with a defective herpes simplex virus 1 vector results in stable expression of Escherichia coli beta-galactosidase. Proc Natl Acad Sci USA. 1990 February;87(3):1149-53. PMID: 2153970
71. Le Gal La Salle et al. An adenovirus vector for gene transfer into neurons and glia in the brain. Science. 1993 Feb. 12; 259(5097):988-90. PMID: 8382374
72. Davidson et al. A model system for in vivo gene transfer into the central nervous system using an adenoviral vector. Nat Genet. 1993 March;3(3):219-23. PMID: 8387378
73. Yang et al. Cellular and humoral immune responses to viral antigens create barriers to lung-directed gene therapy with recombinant adenoviruses. J Virol. 1995 April; 69(4):2004-15. PMID: 7884845
74. Kaplitt et al. Long-term gene expression and phenotypic correction using adeno-associated virus vectors in the mammalian brain. Nat Genet. 1994 October;8(2):148-54. PMID: 7842013
75. Avid et al. Direct gene transfer into skeletal muscle in vivo: factors affecting efficiency of transfer and stability of expression. Hum Gene Ther. 1993 April; 4(2):151-9. PMID: 8494924
76. Quentin et al. Adenovirus as an expression vector in muscle cells in vivo. Proc Natl Acad Sci USA. 1992 Apr. 1; 89(7):2581-4. PMID: 1557362
77. Stratford-Perricaudet et al. Widespread long-term gene transfer to mouse skeletal muscles and heart. J Clin Invest. 1992 August; 90(2):626-30. PMID: 1644927
78. Rosenfeld et al. In vivo transfer of the human cystic fibrosis transmembrane conductance regulator gene to the airway epithelium. Cell. 1992 Jan. 10; 68(1):143-55. PMID: 1370653
79. Mannino et al. Liposome mediated gene transfer. Biotechniques. 1988 Jul-Aug;6(7):682-90. PMID: 2856201
80. Joshi et al. Identification of protein interaction antagonists using the repressed transactivator two-hybrid system. Biotechniques. 2007 May; 42(5):635-44. PMID: 17515203
81. Hirst et al. A two-hybrid system for transactivator bait proteins. Proc Natl Acad Sci USA. 2001 Jul. 17; 98(15):8726-31. PMID: 11447261
82. Fields et al. A novel genetic system to detect protein-protein interactions. Nature. 1989 Jul. 20; 340(6230):245-6. PMID: 2547163
83. Luo et al. Mammalian two-hybrid system: a complementary approach to the yeast two-hybrid system. Biotechniques. 1997 February;22(2):350-2. PMID: 9043710

What is claimed is:

1. A method of preventing and treating viral infections in animals, including the steps of:
inhibiting viral entry protein-to-cellular receptor interaction in an animal, and preventing and treating the viral infection in the animal.

2. The method of claim 1, wherein the treating step is further defined as a step chosen from the group consisting of (non-) or competitive inhibition of viral ligand-cellular receptor interactions through engineered antibody therapeutics; virus neutralization by engineered antibody therapeutics; virus neutralization by engineered antibody therapeutic that also prevent phagocytosis and endo/pinocytosis (CD47 domain included in the Fc region of the antibody); virus neutralization by engineered antibody therapeutics with bispecific heavy and light chain epitopes; virus neutralization by engineered antibody therapeutics with bispecific heavy and light chain epitopes that also prevent phagocytosis and macropinocytosis (CD47 domain included in the Fc region of the antibody); (non-) or competitive inhibition of the viral ligand-cellular receptor interactions with small molecules; cellular receptor altering through gene editing methods so that the viral entry proteins no longer recognize a natural/wildtype receptor; and combinations thereof.

3. The method of claim 1, wherein the preventing step is further defined as a step chosen from the group consisting of immune stimulation (B-cell) through the injection of viral proteins or domains of the proteins that are involved with ligand-cellular receptor interactions; immune stimulation (T-cell) through the injection of viral T-cell antigens; immune stimulation (B-cell and T-cell simultaneously) through the injection of viral proteins or domains of the proteins that are involved in the ligand-cellular receptor interaction or T-cell antigens; the delivery of mRNA encoding viral proteins or domains of the proteins that are involved in ligand-cellular receptor interactions to elicit an immune response from B-cells to produce neutralizing antibodies; and combinations thereof.

4. The method of claim 1, wherein the viral infection is African swine fever virus (ASFV).

5. The method of claim 1, wherein the viral infection is chosen from the group consisting of Pseudorabies virus, Bluetongue virus, Foot-and-mouth disease virus (serotypes A, O, C, SAT1, SAT2, SAT3, Asia1), Japanese encephalitis virus, Rabies virus, Rift Valley fever virus, Rinderpest virus, Vesicular stomatitis virus, West Nile fever virus, BSE prion, Bovine viral diarrhea virus, Bovine leukemia virus, Bovine herpesvirus 1, Lumpky skin disease virus, Caprine arthritis and encephalitis virus, Peste-des-petits-ruminants virus, Scrapie prion, Sheeppox and goatpox viruses, African horse sickness virus, Eastern equine encephalomyelitis virus, Western equine encephalomyelitis virus, Equine infectious anemia virus, Equine influenza virus, Equine herpesvirus 4, Equine arteritis virus, Venezuelan equine encephalomyelitis virus, Classical swine fever virus, Nipah virus, Porcine reproductive and respiratory syndrome virus, Swine vesicular disease virus, Transmissible gastroenteritis virus of swine, Avian infectious bronchitis virus, Infectious laryngotracheitis virus, Duck hepatitis virus, High and low pathogenic avian influenza viruses, Infectious bursal disease virus, Marek's disease virus, Newcastle disease virus, and Avian metapneumovirus.

6. The method of claim 1, further including, before said inhibiting step, the step of performing receptor screening and identifying cellular receptors that interact with viral attachment and entry proteins.

7. The method of claim 1, wherein said cellular receptor altering through gene editing methods further includes the steps of preventing virus binding through dysfunction or disruption of entry proteins.

8. The method of claim 7, wherein the gene editing methods use nucleases chosen from the group consisting of Zinc finger nuclease, transcription activator-like effector nuclease, human WRN, C2c2, C2c1, C2c3, CRISPR Cas9, CRISPR/Cpf1. CRISPR/TevCas9, CasX, CasY, and Archaea Cas9.

9. A method of treating a viral infection in an individual with a virus that is both lysogenic and lytic, including the steps of:
administering a viral antigen that targets protein on an outer membrane of a lysogenic phase of the virus;
administering a viral antigen that targets protein on a capsid of a lytic phase of the virus; and
treating the viral infection.

10. The method of claim 9, wherein the viral infection is ASFV and wherein the individual is a swine.

11. The method of claim 9, wherein said administering a viral antigen that targets protein on an outer membrane of a lysogenic phase of the virus step is further defined as targeting the following proteins (and their variants from all viral genotypes/serogroups); CD2v (EP402R) and/or, EP153R and/or, I177L and/or p12 (061R), but not limited to these lysogenic-linked proteins.

12. The method of claim 9, wherein said administering a viral antigen that targets protein on a capsid of a lytic phase of the virus step is further defined as targeting a protein chosen from the group consisting of (and their variants from all viral genotypes/serogroups); p14.5 (E120R) and/or p72 (B646L) and/or, p49 (B438L) and/or, p54 (E183L) and combinations thereof, but not limited to these lytic-linked proteins.

13. The method of claim 9, wherein said administering a viral antigen that targets protein on a capsid and is secreted from the infected cell as soluble target of both the lysogenic and lytic phase of the virus step is further defined as targeting the protein (and its variants from all viral genotypes/serogroups); p30 (CP204L) that functions to mediate cell receptor and viral ligand or membrane interactions, but limited to this structural and host cell secreted protein.

14. The method of claim 9, wherein each of said administering steps include administering a composition chosen from the group consisting of whole protein, a peptide, peptide segments, and a mixture of peptides derived from target proteins.

15. The method of claim 14, wherein the composition is derived from a protein chosen from the group consisting of (and their variants from all viral genotypes/serogroups); CD2v (EP402R), EP153R, I177L, p54 (E183L), p14.5 (E120R), p72 (B646L), p30 (CP204L), p49 (B438L), p12 (061R), p11.5 (Al 37R) and combinations thereof.

16. The method of claim 9, wherein said administering steps are performed with a single injection or separate injections.

17. The method of claim 9, wherein said treating step further includes the step of inducing a B-cell response in the individual and creating an immune stimulating response.

18. A composition for treating a viral infection in an individual with a virus that is both lysogenic and lytic comprising a viral antigen that targets protein on an outer membrane of a lysogenic phase of said virus and a viral antigen that targets protein on a capsid of a lytic phase of said virus.

19. The composition of claim 18, wherein said viral infection is ASFV and wherein said individual is a swine.

20. The composition of claim 18, wherein said protein (and their variants from all viral genotypes/serogroups) on an outer membrane of a lysogenic phase of said virus is further defined as CD2v (EP402R), EP153R,1177L, p12 (061R).

21. The composition of claim 18, wherein said protein on a capsid of a lytic phase of the virus is further defined as a protein chosen from the group consisting of (and their variants from all viral genotypes/serogroups); p14.5 (E120R), p72 (B646L), p49 (B438L), p11.5 (A137R) and combinations thereof.

22. The composition of claim 18, wherein said protein on a capsid and is secreted from the infected cell as soluble target of both the lysogenic and lytic phase of the virus step is further defined as targeting the protein p30 (CP204L) (and its variants from all viral genotypes/serogroups) that functions to mediate cell receptor and viral ligand or membrane interactions.

23. The composition of claim 18, wherein said composition includes viral antigens chosen from the group consisting of whole protein, a peptide, peptide segments, and a mixture of peptides derived from said target proteins.

24. The composition of claim 23, wherein said viral antigens are derived from a protein chosen from the group consisting of (and their variants from all viral genotypes/serogroups); CD2v (EP402R), EP153R, p54 (E183L), I177L, p14.5 (E120R), p72 (B646L), p30 (CP204L), p49 (B438L), p12 (061R), p11.5 (A137R) and combinations thereof.

25. The composition of claim 18, wherein said composition is formulated with pharmaceutically acceptable excipients in a single injection.

26. The composition of claim 18, wherein said composition is formulated with pharmaceutically acceptable excipients with said viral antigen that targets protein on an outer membrane of a lysogenic phase in a first injection and said viral antigen that targets protein on a capsid of a lytic phase in a second injection.

27. A vaccine for preventing viral infection, comprising whole and/or partial domains of proteins of both a lysogenic and lytic phase of a virus.

28. The vaccine of claim 27, wherein said proteins are chosen from the group consisting of (and their variants from all viral genotypes/serogroups); CD2v (EP402R), EP153R, I177L, p54 (E183L), p14.5 (E120R), p72 (B646L), p30 (CP204L), p49 (B438L), p12 (061R), p11.5 (A137R) and combinations thereof.


SEQUENCE LISTING (2 sequences)

SEQ ID NO: 1 - DNA, 2759 bp, Sus scrofa
ggcacgaggggcaccgggtcctgcctttgacgggaggaggcggcgggtcctctctgtgacggcggcggtgcctgctccagacacctgaggcggcggcggcagccccgcggcggacgcggagatgtggcccttggtggtggtggtgctgctgggctcggcgtactgcggttcagctcagttgatatttaatataaccaaatctgtagaattcaccgtttgcaatacaactgttaccatcccatgttttgttaataacatggaggcaaagaacatttctgaattgtatgtcaagtggaaatttaaaggaaaagacattttcatctttgatggcgctcagcacatatccaagcccagtgaagccttccctagttcaaaaatctcaccatcagaattactacatggcattgcctctttgaagatggataagcgtgatgccgtcataggaaactacacttgtgaagtaacagaattaagcagagaaggcgaaaccatcatagaactaaaacgtcgttttgtttcatggttttctccaaatgaaaatatcctcattgttattttcccaattttggctatacttctgttctggggacagtttggtattttgactcttaaatataaatccagttatacgaaggagaaaaccatttttctattagttgctggactaatgctcactatcattgtcattgttggagccattcttttcatcccaggtgaatattcaacaaagaatgcttgtggacttggtttaattgtaatccctacagcaatattaatattacttcaatactgtgtgtttatgatggctcttggaatgtcttccttcaccattgccatattgatccttcaggtattgggccatgtgctctctgtggttggacttagcctctgcgtctcagagtgtaccccagtgcatggccctcttctgatttcaggtttgggtattatagctctagcagaattacttggactagtttatatgaaatgtgttgcttctgatcacaagactatacaacctcctaggaataactgaagtgaagtgatggactctgatttggagagtactaagacgtgaaagatacacacttgtgtttaagcaccacggccttgattcactgtttgggagaagaaaataagaagagtaactggtttccatctatgggagaaagtaaatcattgacctttaaattgttacagtttaagtttttattcaaagcatttttaatttagttaataaaattacaatctgtgttgtgtgcccaattgagatccagttctttgttgttatttttattcaattatggacaaaagcagagtgggcaacttccaagaatgatacctttcagatcctggggtttcttgaccctaggtcaccaggttaaattgattcaaggtgagaattcctggcactaacctggttacccagtggtatctctgaaaacggtggctttcattgaaccttagcaaactgagtttgcagccactttggcagttatggccttaacctgagtaatgtgtctgctgctcctgggtcaagggaataacaaattgagtacacacagaaatttggttgggaactgcctacatggaatgtgctactgatggggatcgtggtcttcattcttgggggctgccactcacacgttattccttcaacatgcaatgtaccaggccctttccagattgagggtaactttactgatggatgtgttctttttttcacacaaccctttacatcctgtcttgtcctcctgttatttgcttctgctgtacaagatgtagcactttttctcctctttgaacatggtccagtgacacggtagcatcactgcagaaaggagccagacttattctcagagaactatgttcacacttttcagcaaaaatagcgatggttgtaacatatgtattcccttcctcggatttgaaggcacaatctacagtgtttctttgcttcttttctgatctggggcatgaaaaactaagattgagatttgaactgtgagtctcctgcatggcaacataacgtgtgtcactgtcaggccagtaggccggccctgcaagggtggttttattactgttgtatctgtgttgcatgataagcacccatcatcttcctcctgtagtcctgccttgtattcaccttactcaaagattgaaaagtgaaacaaaacccccatgtttcctagccccccccgcccctgttagaagaaaattaacattctgacagttgtgatcgcctgaagaacttttagattattagcgctcgttttcttcccttgtgggtgtgtgtttctatgtgcacctgtgtaagttaggcacatgcatcttctgtatggatgacagcggggtacctacaggagagcaaatgttaatttcatgcttctagtaaaaacatttaaagacagttctttattgggtggacttatatttgatgtgaatatttgatcacttaaaactttaaaaaattctaggtaatttgccaagcattttgactgctcaccgataccctctaaaaatactagttattctccctgtttgtgtaataaaatcttcgtatgtgtagttgcattattaatagttatttcttagtccactgaatgtccccatgtgcctcttttatgccaaattacttgttatattttattctgggaccaagtggtttgctgcagcaaaccaaaatttttgacccgctgatgcctctcagaaaagtaaacacactatgaagatagctcttcttgaaaaaaaaaaaaaaaaaa

SEQ ID NO: 2 - protein, 302 aa, Sus scrofa
MWPLVVVVLLGSAYCGSAQLIFNITKSVEFTVCNTTVTIPCFVNNMEAKNISELYVKWKFKGKDIFIFDGAQHISKPSEAFPSSKISPSELLHGIASLKMDKRDAVIGNYTCEVTELSREGETIIELKRRFVSWFSPNENILIVIFPILAILLFWGQFGILTLKYKSSYTKEKTIFLLVAGLMLTIIVIGAILFIPGEYSTKNACGLGLIVIPTAILILLQYCVFMMALGMSSFTIAILILQVLGHVLSVVGLSLCVSECTPVHGPLLISGLGIIALAELLGLVYMKCVASDHKTIQPPRNN